(19) United States
(12) Patent Application Publication
(10) Pub. No.: US 2010/0222228 A1
(43) Pub. Date: Sep. 2, 2010

(54) COMPOSITIONS AND METHODS FOR TREATING AND DIAGNOSING IRRITABLE BOWEL SYNDROME

(75) Inventors: Theodoor Victor Constant Thielemans, Beerse (BE); Jeroen Marcel Maria Roger Aerssens, Nieuwrode (BE); Willem Jan-Paul Edmond Talloen, Antwerpen (BE); Leen Albert Leonarda Thielemans, Vosselaar (BE); Michael L. Camilleri, Rochester, MN (US)

Correspondence Address: WOODCOCK WASHBURN LLP, CIRA CENTRE, 12TH FLOOR, 2929 ARCH STREET, PHILADELPHIA, PA, 19104-2891, US

(73) Assignees: JANSSEN PHARMACEUTICA N.V., Beerse (BE); MAYO FOUNDATION FOR MEDICAL EDUCATION AND RESEARCH, Rochester, MN (US)

(21) Appl. No.: 12/445,144

(22) PCT Filed: Oct. 11, 2007
(86) PCT No.: PCT/EP2007/008872

§ 371 Date: Apr. 10, 2009

Related U.S. Application Data
(60) Provisional application No. 60/850,659, filed on Oct. 11, 2006.

(30) Foreign Application Priority Data
Nov. 14, 2006 (EP) 06023597.5

(51) Int. Cl. C40B 30/04; C12Q 1/68; G01N 33/53

(52) U.S. Cl. 506/9; 435/6; 435/7.1

(57) ABSTRACT

The present invention relates generally to therapy and diagnosis of disorders associated with chronic visceral hypersensitivity (CVH), and in particular irritable bowel syndrome (IBS). In particular, this invention relates to the polypeptides as well as to the polynucleotides encoding these polypeptides, wherein said polypeptides are shown to be associated with CVH. These polypeptides and polynucleotides are useful in the diagnosis, treatment and/or prevention of disorders associated with CVH, in particular in the diagnosis, treatment and/or prevention of disorders associated with IBS.

The present invention relates generally to therapy and diagnosis of disorders associated with chronic visceral hypersensitivity (CVH), and in particular irritable bowel syndrome (IBS). In particular this invention relates to the polypeptides as well as to the polynucleotides encoding these polypeptides, wherein said polypeptides are shown to be associated with CVH. These polypeptides and polynucleotides are useful in the diagnosis, treatment and/or prevention of disorders associated with CVH, in particular in the diagnosis, treatment and/or prevention of disorders associated with IBS.

### BACKGROUND OF THE INVENTION

Irritable bowel syndrome (IBS) and inflammatory bowel disease (IBD) represent two conditions characterized by chronically recurring symptoms of abdominal pain, discomfort (urgency and bloating) and alterations in bowel habits. However, these bowel diseases differ significantly in etiology and physiopathology, thus implicating different methods of diagnosis and treatment. Where IBD is characterized by inflammation or ulcerations in the small and/or large intestine, such overt structural changes have not been associated with IBS. IBS is classified as a functional (opposed to an organic) bowel disorder of unknown etiology. A functional disorder refers to a disorder or disease where the primary abnormality is an altered physiological function, rather than an identifiable structural or biochemical cause. Diagnosis of IBS is currently based on a characteristic cluster of symptoms in the absence of detectable structural abnormalities (Drossman D A, Camilleri M, Mayer E A, Whitehead W E. AGA technical review on irritable bowel syndrome. Gastroenterology. 2002; 123:2108-31), including intermittent abdominal pain and discomfort and alterations in bowel habits, such as loose or more frequent bowel movements, diarrhea, and/or constipation that occur in the absence of detectable ongoing organic disease. Because of the lack of specificity of the cardinal symptoms of abdominal pain or abdominal discomfort, the current diagnosis of IBS applies to a heterogeneous group of patients, even after attempts to define subgroups based on predominant bowel habit (diarrhea-predominant, constipation-predominant, alternating diarrhea and constipation, normal). IBS affects approximately 10-20% of the general population. It is the most common disease diagnosed by gastroenterologists and one of the most common disorders seen by primary care physicians.

Current theories to explain the pathophysiology of IBS include alterations in visceral perception, gastrointestinal motility or gut epithelial and immune function. Published evidence demonstrate that IBS is associated with a state of chronic visceral hypersensitivity (CVH) suggesting that processing of visceral sensory information is altered, but the molecular changes underlying the development and maintenance of CVH in IBS are not known. Published evidence supports a role of psychosocial and physical (e.g. enteric infections) stressors as central and peripheral triggers, respectively, which may induce presentation or exacerbation of IBS symptoms. There is for example increasing evidence of a putative role of low-grade chronic inflammation in the pathogenesis of IBS. As a consequence, current medical treatments for IBS primarily target peripheral symptoms rather than the underlying causes, and therapeutic gains from drug treatments are usually modest and the placebo responses are high (Mertz H, Naliboff B, Munakata J, Niazi N, Mayer E A. Altered rectal perception is a biological marker of patients with irritable bowel syndrome. Gastroenterology. 1995; 109: 40-52). Defining the underlying neurological and molecular defects is therefore important to the design of more successful therapeutic strategies. Moreover, there is a need in the art for improved methods for screening, diagnosing and treating IBS and other CVH-related disorders.

In a first effort to try and identify the underlying molecular defects, Pasricha P et al. (PCT publication WO 2005/020902) report the analysis of microarray expression profiles of colon tissue RNA and S1 dorsal root ganglia RNA from a rat model of chronic visceral hypersensitivity upon treatment with CNI-1493.

Here the analysis of microarray expression profiles of sigmoid colon mucosal biopsies from IBS patients and healthy subject control subjects is reported. This analysis revealed a number of differentially expressed genes in IBS patients that point to functional alterations of specific components of the host defense system and the immune response. This is in support of an important role for peripheral gastrointestinal changes underlying the aetiology of IBS. Two gene probe sets with the most strikingly increased expression in mucosal colon biopsies of IBS patients represent a gene that is, as yet, uncharacterised (DKFZP564O0823). It is proposed to rename this gene IBS1. The identification of specific sets of gene probes on the microarray, so-called molecular signatures that enable the distinction of IBS patients from healthy control subjects is described. The expression profiles in IBS are consistent across different locations within the colon and are stable over time. Therefore, the identified molecular signatures provide the opportunity to develop biomarkers that are of use in the diagnosis and assessment of the response to therapy in (subsets of) IBS patients. This represents a significant advance based on specific changes in biological activities rather than the current standard, which depends exclusively on the change in clinical symptoms only.

### SUMMARY OF THE INVENTION

The present invention is based on the surprising finding that, despite the absense of structural abnormalities in colon of IBS patients, it is not only possible to identify differentially expressed genes compared to normal tissue but that genes of which the differential expression is associated with IBS have a diagnostic, predictive and/or therapeutic value.

Accordingly, the present invention relates to the identification of a number of genes that were hitherto not associated with IBS, hereinafter referred to as IBS molecular signature genes (IBS-MSGs) (Table 1), and accordingly provides nucleic acid molecules and proteins related to said genes and the use thereof in methods to identify compounds, which may be used in the treatment of CVH, in particular in the treatment of IBS or in diagnostic methods to identify and monitor IBS in a subject.

The nucleic acid molecules can be used individually, e.g. to monitor the level of expression of an individual gene, or they can be provided in a microarray format, to identify and/or monitor IBS in a subject. The nucleic acid molecules can be used to design antisense oligonucleotides and short-interferring RNA (siRNA), ribozymes and other molecules useful for modifying gene expression, for diagnostic, screening and therapeutic purposes. Furthermore, the nucleic acid molecules can be used to express the encoded proteins. One skilled in the art can also design peptide antigens based on the nucleic acid sequences.

The proteins are useful as targets for drug discovery, e.g. to identify lead compounds that agonize or antagonize their activity, as described below. In addition, the proteins can be used to generate antibodies or other specific binding agents. These specific binding agents may be used in methods for treating, diagnosing or monitoring IBS in a subject or in methods for screening, i.e. to identify compounds that may be used in the treatment of CVH, in particular in the treatment of IBS.

In one aspect, the present invention provides methods, more particularly in vitro methods, for diagnosing and monitoring CVH and in particular IBS, by comparing the expression levels of one or more of the IBS-MSGs at the nucleotide or protein level in biological samples from a subject to control samples.

In one embodiment, the present invention provides methods for detecting and/or monitoring IBS in a subject, which methods comprise the steps of (a) determining, in a biological sample of said subject, the level of gene transcription of an IBS-MSG; (b) comparing the level of gene transcription with the level of gene transcription in a normal control sample; and (c) producing a diagnosis based on the result from step (b). The IBS-MSG is typically selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20. More particularly the methods involve determining that there is a difference in expression of these genes compared to expression of these genes in a control sample, whereby a difference in expression in one or more of these genes is indicative of IBS, the status of IBS and/or the susceptibility to a specific type of treatment.

In particular embodiments of the methods of the present invention, step a) includes determining two, three, four, five, six, seven, eight or more of the IBS-MSGs listed above. In particular step (a) consists of determining the expression levels of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; more in particular step (a) consists of determining the expression levels of IBS1, COP1, PSME2, F13A1, NCF4, CSF1R, M160, KCNS3 and VSIG2; alternatively it consists of determining the expression levels of IBS1, PSME2, F13A1, NCF4, CSFR1 and VSIG2; alternatively, it consists of determining the expression levels of MUC20, VSIG2 and VSIG4.

In particular embodiments of the methods of the present invention, step a) includes determining two, three, four, five, six, seven, eight or more of the IBS-MSGs selected from the group consisting of IBS1, COP1, PSME2, F13A1, NCF4, CSF1R, M160, KCNS3, VSIG2; alternatively two, three, four, five, six from the group consisting of IBS1, PSME2, F13A1, NCF4, CSFR1 and VSIG2; alternatively two or three selected from the group consisting of MUC20, VSIG2 and VSIG4.

In particular embodiments of the methods of the present invention, step a) further includes determining the level of gene transcription of at least one, two, three or more other genes, in particular selected from the group consisting of CASP1, FCGR2A and CKB.

The invention further provides methods for identifying or determining IBS in a subject, said method comprising the steps of (a) determining, in a biological sample of said subject, the level of gene transcription of an IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; and (b) comparing the level of gene transcription with the level of gene transcription in a normal control sample and determining whether or not the sample is indicative of IBS, indicative of the status of IBS and/or indicative of the susceptibility to a specific type of treatment; wherein an increase in the level of gene transcription of a gene selected from the group consisting of IBS1, VSIG2 and MUC20 or a decrease in the level of gene transcription of a gene selected from the group consisting of COP1, PSME2, F13A1, NCF4, CSF1R, M160, KCNS3, LYZ, MS4A4A, HELLS, RFC4, MCM5, TAP2, LRAP, DTL and VSIG4, is an indication of IBS in said subject. Thus, differences in the level of gene transcription and, more particularly, an increase in the level of gene transcription of a gene selected from the group consisting of IBS1, VSIG2 and MUC20 or a decrease in the level of gene transcription of a gene selected from the group consisting of COP1, PSME2, F13A1, NCF4, CSF1R, M160, KCNS3, LYZ, MS4A4A, HELLS, RFC4, MCM5, TAP2, LRAP, DTL and VSIG4, is an indication of the presence of IBS in said subject.

The methods of the present invention involve determining the increase and/or decrease of expression of particular genes. In particular embodiments the methods involve determining whether there is an increase corresponding to at least a 1.15, more particularly at least a 1.2 fold change or whether there is a decrease corresponding to at least a 0.85 fold change, more particularly at least an 0.8 fold change in expression of the gene compared to controls.

In step (a) of the methods of the invention the level of gene transcription is determined either at the protein level, preferably using antibodies that bind to the IBS-MSG polypeptide, or at the gene transcription level, preferably using probes that specifically bind to an oligonucleotide transcribed from said IBS-MSG, preferably at the cDNA or mRNA level. The level of gene transcription is optionally determined using array technology, either at the oligonucleotide level using specific probes as described herein or at the protein level using specific binding agents, preferably antibodies as described herein.

In specific embodiments of the methods of the invention, the level of gene transcription is assessed using an array of oligonucleotide probes that bind to the IBS-MSGs. Optionally, the arrays of oligonucleotide probes for the IBS-MSGs are combined with probes that specifically bind to other genes, in particular selected from the group consisting of CASP1, FCGR2A and CKB.

Further specific embodiments relate to methods wherein the expression levels of the IBS genes are determined using an array of the probes enlisted in Table 1, more in particular using an array of the probes enlisted in Table 2.

According to another specific embodiment, the level of gene transcription is assessed using reverse-transcription quantitative polymerase chain reaction (RTQ-PCR).

In specific embodiments of the methods involving detecting expression level of the IBS genes, the biological sample used in the methods of the present invention is selected from the group consisting of blood (including total blood, serum, plasma and in particular white blood cells), urine, saliva, fecal sample, fecal cells, tissue biopsy (in particular colon biopsy) or autopsy material.

Further embodiments of the methods of the invention provide methods for identifying and/or monitoring IBS in a subject said method comprising the steps of (a) determining, in a biological sample of said subject, the protein level of at least one IBS-MSG protein; (b) comparing the protein level with the protein level in a normal control sample; and (c) determining whether or not the sample is indicative of IBS and/or producing a diagnosis based on the result from step (b). Thus, according to these embodiments, the level of gene transcription of an IBS-MSG is determined at the protein level. Typically, in these methods, the protein level is determined using an antibody that binds to an IBS-MSG protein. In specific embodiments the antibody is an antibody to a polypeptide or protein encoded by an IBS-MSG selected from IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20. In a most particular embodiment the protein level of the IBS1 protein is determined using an antibody specific for the gene product of IBS1.

In specific embodiments, the methods encompass determining the protein level of at least two, optionally three, four, five, six or more IBS-MSG proteins, from the proteins listed above. In particular the methods encompass determining the protein level of the peptides or proteins encoded by IBS-MSGs of the group consisting of IBS1, COP1, PSME2, F13A1, NCF4, CSF1R, M160, KCNS3, and VSIG2; Alternatively the IBS-MSGs of the group consisting of IBS1, PSME2, F13A1, NCF4, CSFR1 and VSIG2; Alternatively, the IBS-MSGs of the group consisting of MUC20, VSIG2 and VSIG4.

In particular embodiments, the invention provides methods for detecting and/or monitoring IBS in a subject, said method comprising (a) contacting a biological sample of said subject with an agent that specifically binds with an IBS-MSG polypeptide; (b) determining the level of binding of the agent to the polypeptide; and (c) comparing the level of binding of the agent in said biological sample with the level of binding of the agent in a normal control sample; and (d) producing a diagnosis (or determining whether or not the sample is indicative of IBS) based on the result of step (c). Step (d) typically includes determining whether or not the sample is indicative of IBS or a particular status of IBS based on the result of step (c). Optionally, in such methods the level of binding is determined using a protein array of IBS-MSG specific antibodies. Again, specific embodiments relate to methods wherein the IBS-MSG specific antibodies are reactive with IBS-MSGs selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20. In particular embodiments, the methods encompass using at least two binding agents, each reactive with a different IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20. More particularly, the method encompasses detecting two, three, four, five, six or more binding agents selected from this group. In particular embodiments of these methods the IBS-MSGs that are detected consist of IBS1, COP1, PSME2, F13A1, NCF4, CSF1R, M160, KCNS3 and VSIG2; more in particular the IBS-MSGs that are detected consist of IBS1, PSME2, F13A1, NCF4, CSFR1 and VSIG2; even more in particular the IBS-MSGs that are detected consist of MUC20, VSIG2 and VSIG4. According to an alternative particular embodiment, detecting the IBS-MSG comprises determining the protein level of the polypeptide encoded by IBS1 with specific antibodies reactive with the IBS1 polypeptide. According to yet a further particular embodiment, detecting the IBS-MSG consists of determining the protein level of the polypeptide encoded by IBS1 with specific antibodies reactive with the IBS1 polypeptide.

Specific embodiments of the methods of the present invention make use of protein arrays, wherein the protein arrays for the IBS-MSG are optionally combined with other known IBS markers, in particular selected from the group consisting of CASP1, FCGR2A and CKB.

Specific embodiments of the methods of the present invention encompass methods wherein an increase in the level of binding of the specific binding agent for an IBS-MSG gene selected from the group consisting of IBS1, VSIG2 and MUC20 or a decrease in the level of binding of the specific binding agent for an IBS-MSG gene selected from the group consisting of COP1, PSME2, F13A1, NCF4, CSF1R, M160, KCNS3, LYZ, MS4A4A, HELLS, RFC4, MCM5, TAP2, LRAP, DTL and VSIG4, is an indication of IBS in said subject.

In specific embodiments of the methods involving detecting the binding of an agent to an IBS-MSG polypeptide, the biological sample is selected from the group consisting of blood (including total blood, serum, plasma and in particular white blood cells), urine, saliva, fecal sample, fecal cells, tissue biopsy (in particular colon biopsy) or autopsy material.

Another aspect of the present invention provides methods for screening anti-CVH, in particular anti-IBS agents based on the agent's interaction with the IBS-MSG products, or the agent's effect on the activity or expression of said IBS-MSG/IBS-MSG products.

Accordingly, the present invention provides methods, in particular in vitro methods, for identifying a candidate compound for the treatment of CVH, in particular for the treatment of IBS, the method comprising the steps of (a) contacting a cell expressing at least one IBS-MSG with the compound to be tested; (b) determining the expression level of said IBS-MSG; and (c) comparing with the expression level of said IBS-MSG in the absence of said compound; whereby a compound capable of opposing the change in expression level of the IBS-MSG observed in IBS, is identified as a candidate compound for the treatment of CVH, in particular for the treatment of IBS.

Specific embodiments of these methods provide methods wherein the IBS-MSG is selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; In particular, the IBS-MSG used in the screening methods of the present invention consists of IBS1.

Further specific embodiments of these methods encompass contacting a cell expressing at least two IBS-MSGs, determining the expression level of the at least two IBS-MSGs, and comparing the expression level of the at least two MSGs in the absence of the compound. More particularly at two, three, four, five, six or more genes are determined. Particular embodiment encompass determining the expression of the IBS-MSGs corresponding to the group consisting of IBS1, COP1, PSME2, F13A1, NCF4, CSF1R, M160, KCNS3, and VSIG2; more in particular to the group consisting of IBS1, PSME2, F13A1, NCF4, CSFR1 and VSIG2; alternatively, to the group consisting of MUC20, VSIG2 and VSIG4.

In specific embodiments of these methods of the invention the expression level is detected at the nucleic acid level or the protein level. In further particular embodiments, the expression level is determined using a probe which binds to an IBS-MSG, in particular to an IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; most particular the IBS-MSG used in the screening method consists of IBS1. In particular embodiments probes to those IBS-MSGs are used corresponding to the group consisting of IBS1, COP1, PSME2, F13A1, NCF4, CSF1R, M160, KCNS3, VSIG2; more in particular to the group consisting of IBS1, PSME2, F13A1, NCF4, CSFR1 and VSIG2; alternatively, corresponding to the group consisting of MUC20, VSIG2 and VSIG4.

In particular embodiments of these methods of the invention the level of gene expression is determined using a array of oligonucleotide probes that bind to the IBS-MSGs, more in particular using the probes enlisted in Table 1 or using the probe set provided in Table 2.

The invention further provides methods for identifying a candidate compound for the treatment of CVH, in particular for the treatment of IBS, said method comprising the steps of (a) contacting a cell expressing at least one IBS-MSG with the compound to be tested; (b) determining the protein level of said IBS-MSG; and (c) comparing with the protein level of said IBS-MSG in the absence of said compound; whereby a compound capable of opposing the change in protein level of said IBS-MSG observed in IBS, is identified as a candidate compound for the treatment of CVH, in particular for the treatment of IBS. Thus, according to this embodiment, the expression level of the IBS-MSG is determined by determining the protein level of the IBS-MSG polypeptide.

In specific embodiments of the methods of the invention, the protein level is determined using an antibody. In further specific embodiments, the antibody binds to a polypeptide encoded by an IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20. Most particularly, the IBS-MSG used in the screening methods of the present invention consists of IBS1.

In particular, the methods comprise using at least two of the antibodies. More particularly, the methods comprise using two, three, four, five, six of more antibodies, each of which antibody binds to a polypeptide encoded by an IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20. Most particularly the methods involve the detection of the protein level of the gene product of the IBS-MSG of the group consisting of IBS1, COP1, PSME2, F13A1, NCF4, CSF1R, M160, KCNS3, VSIG2; more in particular of the group consisting of IBS1, PSME2, F13A1, NCF4, CSFR1 AND VSIG2; alternatively of the group consisting of MUC20, VSIG2 and VSIG4.

The present invention further provides a screening method to identify and obtain a candidate compound for the treatment of CVH, in particular for the treatment of IBS, said method comprising the steps of (a) incubating an IBS-MSG product with the compound to be tested; and (b) determining the capability of said compound to bind with the IBS-MSG product; wherein a compound capable of binding to the IBS-MSG product is a candidate compound for the treatment of IBS. In these methods, the IBS-MSG product consists of the polypeptide encoded by said gene or a fragment thereof. According to another particular embodiment, the IBS-MSG product is a polynucleotide transcribed from said gene or a fragment thereof.

Yet another aspect of the present invention provides kits for diagnosing CVH, in particular IBS, which kits comprising at least one of the following: (1) a polynucleotide probe that specifically binds to an IBS-MSG, and (2) an agent capable of specifically binding to an IBS-MSG product.

Accordingly, the present invention provides diagnostic kits which comprise: (a) at least one probe that specifically binds to an IBS-MSG; in particular to an IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; or (b) at least one agent that specifically binds to an IBS-MSG polypeptide or a fragment thereof; in particular to an IBS-MSG polypeptide selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; more in particular with an IBS-MSG polypeptide selected from the group consisting of IBS1, COP1, PSME2, F13A1, NCF4, CSF1R, M160, KCNS3 and VSIG2; even more in particular with an IBS-MSG polypeptide selected from the group consisting of IBS1, PSME2, F13A1, NCF4, CSFR1 and VSIG2; most particular with an IBS-MSG polypeptide selected from the group consisting of MUC20, VSIG2 and VSIG4.

In specific embodiments, the kits of the present invention comprise at least two probes or agents, each of which specifically bind to a different IBS-MSG or to an IBS-MSG polypeptide or a fragment thereof, respectively, the IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20. More particular embodiments relate to kits consisting of probes or agents specifically binding to a different IBS-MSG or IBS-MSG product, which correspond to IBS-MSGs of the group consisting of IBS1, COP1, PSME2, F13A1, NCF4, CSF1R, M160, KCNS3 and VSIG2; even more in particular the IBS-MSGs corresponding to the group consisting of IBS1, PSME2, F13A1, NCF4, CSFR1 and VSIG2; Alternatively, the IBS-MSGs corresponding to the group consisting of MUC20, VSIG2 and VSIG4. Kits comprising at least two probes or agents will typically mean containing at least two probes or at least two agents, each of which specifically binds to an IBS-MSG or to an IBS-MSG polypeptide, respectively; but kits comprising at least one probe which specifically binds to an IBS-MSG and at least one agent which specifically binds to an IBS-MSG polypeptide are also envisaged within this terminology.

In yet another aspect, the present invention provides pharmaceutical compositions for the treatment of CVH and in particular IBS. The pharmaceutical compositions comprise a pharmaceutically acceptable carrier and at least one of the following: (1) a IBS-MSG product; (2) an agent that binds with and/or modulates an activity of an IBS-MSG product; and (3) an agent that modulates the expression of a IBS-MSG. It is accordingly an object of the present invention to provide a method for treating CVH and in particular IBS in a patient, said method comprising the step of administering to said patent a pharmaceutical composition as described hereinbefore. In a particular embodiment, the IBS-MSG is IBS1.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1: Concordance correlation analysis of the expression profiles of sigmoid colon samples collected from 10 individuals.
The degree of similarity (concordance correlation coefficient, CCC) of the samples is indicated by color codes. The analysis included three samples for each subject: sample A and B, taken at the same time, located approximately 10 cm away from each other in the colon, and sample C collected 85 days later. Blue squares indicate the concordance correlation analysis for samples from one individual. A thick black line distinguishes the samples from IBS and healthy subjects. The analysis was performed on (A) a set of 1,000 gene probes that showed the largest variation in expression within the full dataset and (B) the set of 32 gene probes identified in the prediction analysis for microarrays.

FIG. 2: Plots representing the results of a spectral map analysis on the microarray data from the sigmoid colon samples of IBS patients and healthy subjects. The different graphs show different combinations of the six first principal components (PC) in the analysis. Red circles represent samples from subjects who volunteered for a repeat mucosal sample; blue circles represent all other subjects (no repeat sample). The subjects who provided a repeat sample did not cluster in any of these graphs, suggesting that they were indeed representative of the entire cohort.

FIG. 3: Schematic overview of the genes differentially expressed in mucosal colon of IBS patients.
Genes with increased (underlined) or decreased expression in mucosal colon samples from IBS patients versus healthy controls. Protein complexes responsible for oxidative burst (2O₂→2O—) are shown as pentagon shapes. “ROS” represent Reactive Oxygen Species

FIG. 4: Relative expression levels of significant genes identified by the Significance of Microarray Analysis (SAM) in mucosal colon samples from IBS patients (green dots) as compared to healthy controls (red dots). Expression levels (y-axis) are expressed as fluorescent signal intensity measured on the array after preprocessing of the raw data (see Methods). Each individual dot represents the averaged expression value of two samples per subject.

FIG. 5: Gene expression profile of DKFZP56400823 (IBS1) and comparative sequence analysis.
(A) Gene expression profiles of two different probe sets (204678_at and 225809_at) on the Affymetrix array that represent DKFZP56400823 (IBS1) in mucosal colon biopsies from IBS patients (green, on the right in the figures) and healthy subjects (red, on the left in the figures). Each circle represents the average of two samples from one individual. Expression levels are expressed as signal intensity measured on the array after preprocessing of the raw data (see Methods). Green and red horizontal lines represent mean expression levels in healthy and IBS subjects, respectively.
(B) This figure shows the excellent correlation between the two probesets that represent the IBS1 gene. Healthy controls and IBS patients are indicated as blank squares and black circles, respectively (see also FIG. 7).
(C) Comparative protein sequence analysis of human (Hs) DKFZP56400823 (IBS1) (Hs_NP_{—}056208) and its mouse (Mm_NP_{—} 663537) and rat (Rn_NP_{—}775137) homologues. Identical amino acids over different species are highlighted with a black or grey background. Different domains of the protein are indicated under the amino acid sequence.

FIG. 6: (A) Examination of DKFZP56400823 gene expression induced by interferon gamma (IFNγ), tumor necrosis factor alpha (TNFα) and interleukin 4 (IL4) inflammatory cytokines on primary colon endothelial cells. From: Gene Expression Omnibus, Accession nr. GDS502 (http://www.ncbi.nlm.nih.gov/projects/geo/gds/profileGraph.cgi?&dataset=MzA&dataset=S4O$&gmin=−0.090309&gmax=−0.032624&gds=502&idref=5543&annot=DKFZP56400823)
(B) Analysis of Jurkat CD4+T cells following induction of simian immune deficiency virus (SIV) Nef from an inducible promoter. The Nef protein is expressed early in HIV and SIV infections and plays a crucial role in disease progression. Results identify Nef-mediated changes in T cell gene expression. From: Gene Expression Omnibus, Accession nr. GDS2164 (http://www.ncbi.nlm.nih.gov/projects/geo/gds/profileGraph.cgi?&dataset=ABPyqz&dataset=LL6gee$&gmin=2.600000&gmax=56.900000&gds=2164 &idref=225809_at&annot=DKFZP56400823)

FIG. 7: Correlation of the expression levels of two probe sets, 204687_at and 225809_at, representing the DKFZP564O0823 gene. Healthy controls and IBS patients are indicated as blank squares (grouped by a dotted line) and black circles (grouped by a dashed line), respectively.

FIG. 8: Summary of the Predictive Analysis of Microarrays (PAM): output from the nearest shrunken centroid classifier on IBS disease status.
(A) The cross-validated misclassification error curve that shows that the lowest misclassification error is obtained when using 32 genes. The corresponding delta (2.0) was selected as threshold for further analysis.
(B) The shrunken class centroids for each class for the 32 genes surviving the threshold (delta=2.0). For more details, see Tibshirani et al (2002).

FIG. 9: Summary of hierarchical clustering analysis.
(A) Clustered display of heatmap with hierarchical clustering of 16 probesets and samples using average linkage and correlation as similarity measure. The colors of the heatmap represent the relative expression level on a color gradient scale ranging from blue (high expression) to black (intermediate expression) to yellow (low expression). This color scale is maximized for each individual probeset over all the samples (i.e., the sample with the highest expression level is blue, and the sample with the lowest expression is yellow). The white horizontal line on the heat map discriminates the disease status as predicted by the obtained molecular signature for IBS. The right panel of the figure shows the clinically diagnosed disease status in the subjects assigned to the training or the test set.
(B) The right panel of FIG. 9A is shown again, now also linked to the gender of the subjects and concomitant drug treatment. (M: male; F: female; SSRI: selective serotonin reuptake inhibitor; SNRI: serotonin-norepinephrine reuptake inhibitor; SNDRI: serotonin-norepinephrine-dopamine reuptake inhibitor; TCA: tricyclic antidepressant).

FIG. 10: Comparison of fold changes in mRNA expression level, as measured by microarray and RTQ-PCR, between IBS patients and healthy subjects. Significant genes from the microarray study that were confirmed statistically significant (p<0.05) in RTQ-PCR analysis are underlined.

### DETAILED DESCRIPTION

The preferred embodiments of the invention are described below. Unless specifically noted, it is intended that the words and phrases in the specification and claims be given the ordinary and accustomed meaning to those of ordinary skill in the applicable art of arts. If any other meaning is intended, the specification will specifically state that a special meaning is being applied to a word or phrase.

It is further intended that the inventions not be limited only to the specific structure, material or acts that are described in the preferred embodiments, but in addition, include any and all structures, materials or acts that perform the claimed function, along with any and all later-developed equivalent structures, materials or acts for performing the claimed invention.

Further examples exist throughout the disclosure, and it is not applicant's intention to exclude form the scope of his invention the use of structures, materials, methods or acts that are not expressly identified in the specification, but nonetheless are capable of performing a claimed function.
As used herein, the term “compound” or “agent” means a biological or chemical compound such as a simple or complex organic molecule, a peptide, a protein or an oligonucleotide. A “test compound” as used herein, refers to a “compound” or “agent” used in a method according to the invention to assess whether said compounds binds with and/or modulates an activity of an IBS-MSG product.
The term “chronic visceral hypersensitivity-molecular signal genes” or “CVH-MSG” as used herein refers to genes, the expression of which is associated with the clinical diagnosis of chronic visceral hypersensitivity (CVH). This includes genes which are specifically upregulated or downregulated in patients diagnosed with CVH compared to healthy control patients.
The term “inflammatory bowel syndrome molecular signature genes” or “IBS-MSG” as used herein refers to genes, the expression of which is associated with the clinical diagnosis of inflammatory bowel syndrome (IBS). This includes genes which are specifically upregulated or downregulated in patients diagnosed with IBS compared to healthy control patients.
The term “IBS-MSG product” or “gene product” as used herein includes a polynucleotide or polypeptide and variants thereof, generated when an IBS-MSG is transcribed and/or translated.
As used herein, a “variant of a polynucleotide” includes a polynucleotide that differs from the original polynucleotide by one or more substitutions, additions, deletions and/or insertions such that the activity of the encoded polypeptide is not substantially changed (e.g., the activity may be diminished or enhanced, by less than 50%, and preferably less than 20%) relative to the polypeptide encoded by the original polynucleotide.
   A variant of a polynucleotide also includes polynucleotides that are capable of hybridizing under reduced stringency conditions, more preferably stringent conditions, and most preferably highly stringent conditions to the original polynucleotide (or a complementary sequence).
   It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotide where alterations are limited to silent changes, i.e. changes that do not alter the amino acids encoded by the polynucleotide are specifically contemplated by the present invention.
   Polynucleotide variants preferably exhibit at least about 70%, preferably at least 80%, more preferably at least 90%, even more preferably at least 95%, in particular at least 97%, and most preferably at least 99% sequence homology with the native polynucleotide. In a further embodiment the polynucleotide variants exhibit at least about 70%, preferably at least 80%, more preferably at least 90%, even more preferably at least 95%, in particular at least 97%, and most preferably at least 99% sequence identity with the native polynucleotide.
The term “hybridization” as used herein refers to a process in which a single-stranded nucleic acid molecule joins with a complementary strand through nucleotide base pairing.
The term “stringency” refers to hybridization conditions. High stringency conditions disfavor non-homologous base pairing. Low stringency conditions have the opposite effect. Stringency may be altered, for example, by temperature and salt concentration. “Stringent conditions” refer to an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulfate, and 20 μg/ml denaturated, sheared salmon sperm DNA, followed by washing the filters in 0.1×SSC at about 65° C. Further suitable hybridization conditions are described in the examples.
“Lower stringency conditions” include an overnight incubation at 37° C. in a solution comprising 6×SSPE (20×SSPE=3M NaCl; 0.2M NaH₂P0₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.
As used herein, a “variant of a polypeptide” is a polypeptide that differs from a native polypeptide in one or more substitutions, deletions, additions and/or insertions, such that the bioactivity or immunogenicity of the native polypeptide is not substantially diminished. In other words, the bioactivity of a variant polypeptide or the ability of a variant polypeptide to react with antigen-specific antisera may be enhanced or diminished by less than 50%, and preferably less than 20%, relative to the native polypeptide. Variant polypeptides include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminal of the mature protein.
   Further variant polypeptides are those which differ from the native polypeptide in amino acid sequence by one or more conservative substitutions. “Conservative substitutions” refers to a replacement of one or more amino acid residue(s) in a parent protein without affecting the biological activity of the parent molecule based on the art recognized substitutability of certain amino acids (See e.g. M. Dayhoff, In Atlas of Protein Sequence and Structure, Vol. 5, Supp. 3, pgs 345-352, 1978).
   A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure, tertiary structure, and hydropathic nature of the polypeptide.
   Polypeptide variants preferably exhibit at least about 70%, more preferably at least about 90%, even more preferably at least 95% and most preferably at least about 97% sequence homology to the original polypeptide. In a further embodiment the polypeptide variants exhibit at least about 70%, more preferably at least about 90%, even more preferably at least 95% and most preferably at least about 97% sequence identity to the original polypeptide.
The terms “complementary” or “complementarity” as used herein refer to the capacity of purine and pyrimidine nucleotides to associate through hydrogen bonding to form double-stranded nucleic acid molecules. The following base pairs are related by complementarity: guanine and cytosine; adenine and thymine; and adenine and uracil. As used herein “complementary” means that the aforementioned relationship applies to substantially all base pairs comprising two single-stranded nucleic acid molecules over the entire length of said molecules. “Partially complementary” refers to the aforementioned relationship in which one of the two single-stranded nucleic acid molecules is shorter in length than the other such that a portion of one of the molecules remains single-stranded.
The term “subject” as used herein refers to a mammal (e.g., a rodent such as a mouse or a rat, a pig, a primate, or a companion animal (e.g., dog or cat)). In particular, the term refers to humans.
The terms “array” and “microarray” are used interchangeably and refer generally to any ordered arrangement (e.g., on a surface or substrate) of different molecules, referred to herein as “probes”. Each different probe of an array is capable of specifically recognizing and/or binding to a particular molecule, which is referred to herein as its “target,” in the context of arrays. Examples of typical target molecules that can be detected using microarrays include mRNA transcripts, cDNA molecules, cRNA molecules, and proteins.
   Microarrays are useful for simultaneously detecting the presence, absence and quantity of a plurality of different target molecules in a sample (such as an mRNA preparation isolated from a relevant cell, tissue, or organism, or a corresponding cDNA or cRNA preparation). The presence and quantity, or absence, of a probe's target molecule in a sample may be readily determined by analyzing whether (and how much of) a target has bound to a probe at a particular location on the surface or substrate.
In a preferred embodiment, arrays used in the present invention are “addressable arrays” where each different probe is associated with a particular “address”. For example, in a preferred embodiment where the probes are immobilized on a surface or a substrate, each different probe of the addressable array is immobilized at a particular, known location on the surface or substrate. The presence or absence of that probe's target molecule in a sample may therefore readily be determined by simply detecting whether a target has bound to that particular location on the surface or substrate.
The arrays according to the present invention are preferably nucleic acid arrays (also referred to herein as “transcript arrays” or “hybridization arrays”) that comprise a plurality of nucleic acid probes immobilized on a surface or substrate. The different nucleic acid probes are complementary to, and therefore can hybridize to, different target nucleic acid molecules in a sample. Thus, such probes can be used to simultaneously detect the presence and quantity of a plurality of different nucleic acid molecules in a sample, to determine the expression level of a plurality of different genes, e.g. the presence and abundance of different mRNA molecules, or of nucleic acid molecules derived therefrom (for example, cDNA or cRNA).
   There are two major types of microarray technology; spotted cDNA arrays and manufactured oligonucleotide arrays. The Examples section below describes the use of high density oligonucleotide Affymetrix GeneChip® arrays. The arrays are preferably reproducible, allowing multiple copies of a given array to be produced and the results, from each easily compared to each other. Preferably the microarrays are small, usually smaller than 5 cm, and are made from materials that are stable under binding (e.g. nucleic acid hybridization) conditions. A given binding site or unique set of binding sites in the microarray will specifically bind the target (e.g., the mRNA of a single gene in the cell). Although there may be more than one physical binding site (hereinafter“site”) per specific target, for the sake of clarity the discussion below will assume that there is a single site. It will be appreciated that when cDNA complementary to the RNA of a cell is made and hybridized to a microarray under suitable hybridization conditions, the level or degree of hybridization to the site in the array corresponding to any particular gene will reflect the prevalence in the cell of mRNA transcribed from that gene. For example, when detectably labeled (e.g. with a fluorophore) cDNA complementary to the total cellular mRNA is hybridized to a microarray, any site on the array corresponding to a gene (i.e. capable of specifically binding a nucleic acid product of the gene) that is not transcribed in the cell will have little or no signal, while a gene for which the encoded mRNA is highly prevalent will have a relatively strong signal. By way of example, GeneChip® expression analysis (Affymetrix, Santa Clara, Calif.) generates data for the assessment of gene expression profiles and other biological assays.
   Oligonucleotide expression arrays simultaneously and quantitatively “interrogate” thousands of mRNA transcripts (genes or ESTs), simplifying large genomic studies. Each transcript can be represented on a probe array by multiple probe pairs to differentiate among closely related members of gene families. Each probe set contains millions of copies of a specific oligonucleotide probe, permitting the accurate and sensitive detection of even low-intensity mRNA hybridization patterns. After hybridization intensity data is captured, e.g., using optical detection systems (e.g., a scanner), software can be used to automatically calculate intensity values for each probe cell. Probe cell intensities can be used to calculate an average intensity for each gene, which correlates with mRNA abundance levels. Expression data can be quickly sorted based on any analysis parameter and displayed in a variety of graphical formats for any selected subset of genes. Gene expression detection technologies include, among others, the research products manufactured and sold by Hewlett-Packard, Perkin-Elmer and Gene Logic.
The term “conservative substitution” or “conservative amino acid substitution” refers to a replacement of one or more amino acid residue(s) in a parent protein without affecting the biological activity of the parent molecule based on the art recognized substitutability of certain amino acids (See e.g. M. Dayhoff, In Atlas of Protein Sequence and Structure, Vol. 5, Supp. 3, pgs 345-352, 1978).
“Fragment thereof” refers to a fragment, piece, or sub-region of a nucleic acid or protein molecule whose sequence is disclosed herein, such that said fragment comprises 5 or more amino acids, or 10 or more nucleotides that are contiguous in the parent protein or nucleic acid molecule.
“Functional fragment” as used herein, refers to an isolated sub-region, or fragment of a protein disclosed herein, or sequence of amino acids that, for example, comprises a functionally distinct region such as an active site for a receptor. Functional fragments may be produced by cloning technology, or as the natural products of alternative splicing mechanism.
The term “homolog” or “homologous” describes the relationship between different nucleic acid molecules or amino acid sequences in which said sequences or molecules are related by partial identity or similarity at one or more blocks or regions within said molecules or sequences. “Isolated nucleic acid compound” refers to any RNA or DNA sequence, however construed or synthesized, which is locationally distinct from its natural location.
As used herein “identity or similarity”, as known in the art, are relationships between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity and similarity between two polynucleotide or two polypeptide sequences, both terms are well known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., (1988) SIAM J. Applied Math., 48, 1073. Methods commonly employed to determine identity or similarity between sequences include, but are not limited to those disclosed in Carillo, H., and Lipman, D., (1988) SIAM J. Applied Math., 48, 1073.
   Methods for comparing the identity and similarity of two or more sequences are well known in the art. Thus for instance, programs available in the Winconsin Sequence Analysis Package, version 9.1 (Devreux J. et al., Nucleic Acid Res., 12, 387-395, 1984), for example the programs BESTFIT and GAP, may be used to determine the % identity between two polynucleotides and the % identity and the % similarity between two peptide or polypeptide sequences. BESTFIT uses the “local homology” algorithm of Smith and Waterman (J. Mol. Biol., 147, 195-197, 1981) and finds the best single region of similarity between two sequences. BESTFIT is more suited to compare two polynucleotide or two peptide or polypeptide sequences that are dissimilar in length, the program assuming that the shorter sequence represents a portion of the longer. In comparison, GAP aligns two sequences, finding a “maximum similarity”, according to the algorithm of Needleman and Wunsch (J. Mol. Biol., 48, 443-453, 1970). GAP is more suited to compare sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned. Other programs for determining identity and/or similarity between sequences are also known in the art, for instance the BLAST family of programs (Altschul S F et al., Nucleic Acids Res., 25:3389-3402, 1997).
A “nucleic acid probe” or “probe” as used herein is a nucleic acid compound, in particular a labeled nucleic acid compound, which hybridizes with another nucleic acid compound. “Nucleic acid probe” means a single stranded nucleic acid sequence that will hybridize with a single stranded target nucleic acid sequence. A nucleic acid probe may be an oligonucleotide or a nucleotide polymer. A “probe” will usually contain a detectable moiety which may be attached to the end(s) of the probe or be internal to the sequence of the probe. In a specific embodiment “probe” is also used to refer to an oligonucleotide, for example about 25 nucleotides in length, attached to a solid support for use on “arrays” and “microarrays” as described hereinbefore.
The term “primer” is a nucleic acid fragment which functions as an initiating substrate for enzymatic or synthetic elongation of, for example, a nucleic acid molecule.
The term “hybridization” as used herein refers to a process in which a single-stranded nucleic acid molecule joins with a complementary strand through nucleotide base pairing.
As used herein, the term “modulation” includes in its various grammatical forms (e.g. “modulated”, “modulation”, “modulating”, etc.), up-regulation, induction, stimulation, potentiation and/or relief of inhibition, as well as inhibition and/or down regulation or suppression.
A nucleic acid sequence is“operably linked” to another nucleic acid sequence when the former is placed into a functional relationship with the latter. For example, a DNA for a presequence or secretory leader peptide is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice.

The present invention is based on the identification of a number of genes which are associated with the clinical symptoms of CVH, more particularly with IBS. These genes have been identified by differential expression analysis of patients diagnosed with IBS and healthy controls. More particularly, the diagnosis of patients with IBS by a gastroentereologist was confirmed by the a bowel disease questionnaire (Talley et al., 1990) including questions to correspond to Rome II criteria (Thompson et al., 1999). The bowel disease questionnaire also includes a psychosomatic symptom checklist intended to identify somatization disorders and symptoms to characterize non-ulcer dyspepsia, and has been used extensively in epidemiological studies. According to the present invention, genes have been identified the expression of which in mucusal colon is either decreased or increased in patients with CVH, more particularly, IBS, compared to healthy controls. A particular advantage of these expression markers is that there is a strong correlation between the expression of particular genes and the occurrence of IBS, and that these expression patterns have a predictive value. Accordingly, these expression markers are useful as a diagnostic tool. Expression markers are not necessarily, and even in most cases, not linked to the presence of a polymorphism in the corresponding genes, distinguishing them from genetic markers.

### Screening Methods

The present invention provides nucleic acid molecules and gene products (proteins) that can be used in screening assays to identify compounds for use as therapeutics for the treatment of CVH, in particular IBS.

The proteins encoded by the nucleic acid molecules described herein can be used in binding and functional assays to screen for lead compounds for treating CVH, in particular IBS. As discussed for each gene product, the ability to identify either an antagonist or agonist would provide for development of new treatments for IBS

Thus, the nucleic acid molecules are useful for expressing the proteins to be isolated and used in direct binding assays. Protein expression can be carried out in any host cell system, e.g, plants, prokaryotes (e.g. E. coli), yeast, insect cells (e.g. Sf9 cells, using baculovirus vectors), or mammalian cells (e.g. CHO, COS etc.). Techniques for the isolation and purification of the protein products are well known to one skilled in the art.

Protein products, or fragments thereof (e.g. proteolytic fragments or synthetic fragments), can be used to generate specific antibodies for directly detecting protein expression, e.g. through immunoassay.

Gene expression profiles may be used in screening for compounds that modulate the mRNA or protein expression of the differentially expressed genes shown in Table 1. Such a differentially expressed gene is referred to as the “gene of interest” and such modulating compounds are referred to as modulators that may up- or down-regulate mRNA transcription, or agonize or antagonize the activity of the protein. Such compounds are useful, e.g. for inhibiting or stimulating the expression of genes found to be regulated in IBS. Compounds that modulate the expression profile of one or more of the genes may be readily identified using numerous screening methods known in the art. As used herein, the expression of a gene can be determined by measuring mRNA levels, protein levels, or protein activity using standard techniques.

It is thus an object of the present invention to provide a method for identifying a candidate compound for the treatment of CVH, in particular for the treatment of IBS, said method comprising;
a) contacting a cell expressing at least one IBS-MSG with the compound to be tested;
b) determining the expression level of said IBS-MSG; and
c) comparing with the expression level of said IBS-MSG in the absence of said compound;
whereby a compound capable of opposing the change in expression level of the IBS-MSG observed in IBS, is identified as a candidate compound for the treatment of CVH, in particular for the treatment of IBS.
The IBS-MSG as used in the screening methods of the present invention is typically selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular from the group consisting of IBS1, COP1, PSME2, F13A1, NCF4, CSF1R, M160, KCNS3, VSIG2; more in particular from the group consisting of IBS1, PSME2, F13A1, NCF4, CSFR1 AND VSIG2; even more in particular from the group consisting of MUC20, VSIG2 and VSIG4; most particular the IBS-MSG used in the screening methods of the present invention consists of IBS1.

According to a particular embodiment, the cell is a colon cell, more particularly, a mucosal colon cell.

According to a particular embodiment, the methods of the invention comprise, in step (b) determining the expression of at least two different genes, one of which is selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20 and one or more other genes is selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20, CASP1, FCGR2A and CKB; in particular determining the expression of at least two genes selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular at least two genes selected from the group consisting of IBS1, COP1, PSME2, F13A1, NCF4, CSF1R, M160, KCNS3, VSIG2; more in particular at least two genes from the group consisting of IBS1, PSME2, F13A1, NCF4, CSFR1 AND VSIG2; even more in particular at least two genes from the group consisting of MUC20, VSIG2 and VSIG4; further embodiments of the present invention comprise in step (b) determining the expression of at least two genes, one of which is IBS1, the other being selected from the group consisting of COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20, CASP1, FCGR2A and CKB.

For each of the embodiments described thereof, step (c) consists of comparing the expression level of said at least two genes by said cells after having contacted said cell with said compound to the expression of said genes by said cells in the absence of said compound.

As used herein, the expression level of an IBS-MSG can be detected at the nucleic acid level or at the protein level. Determining the expression level at the nucleic acid level can be accomplished using any available technology to measure gene transcription levels. For example, the method could employ in situ hybridization, Northern hybridization or hybridization to a nucleic acid microarray, such as an oligonucleotide microarray or a cDNA microarray. Alternatively, the method could employ reverse-transcriptase polymerase chain reaction (RT-PCR) such as fluorescent dye-based quantitative real time PCR (TaqMan® PCR). In the example section provided below, nucleic acid expression levels were obtained by hybridization of labeled cRNA derived from total cellular mRNA to Affymetrix GeneChip® oligonucleotide microarrays and using RTQ-PCR (TaqMan® PCR). The expression levels at the protein level can be assessed using any available technology to measure protein levels. For example, the method could employ protein microarray technology, Western blotting, immunocytochemistry, SDS-PAGE, relative quantification using mass spectrometry and pre-labelling of cells with isotopomeric forms of essential amino acids (Unwin R. D., Evans, C. A. and Whetton A. D. 2006 TRENDS in Biochemical Sciences Vol. 31(8); 473-484).

Preferably, the expression level is determined at the nucleic acid level. In this instance mRNA or cDNA may be used directly for detection or may be amplified enzymatically using PCR or other amplification techniques prior to analysis. Preferably said analysis methods comprise the use of a labelled oligonucleotide probe targeted to a suitable region of the polynucleotide. Accordingly in a particular embodiment the expression level is determined using a probe which binds to an IBS-MSG, in particular to an IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular from the group consisting of IBS1, COP1, PSME2, F13A1, NCF4, CSF1R, M160, KCNS3, VSIG2; more in particular from the group consisting of IBS1, PSME2, F13A1, NCF4, CSFR1 AND VSIG2; even more in particular from the group consisting of MUC20, VSIG2 and VSIG4; most particular the IBS-MSG used in the screening method consists of IBS1. In an even further embodiment the level of gene expression is determined using an array of oligonucleotide probes that bind to the IBS-MSGs, more in particular using the probes enlisted in Table 1; most in particular using the probe set provided in Table 2 below.

TABLE 2
DESCRIPTION OF THE PROBE SETS

CASP1 specific probes
caspase 1, apoptosis-related cysteine peptidase (interleukin 1, beta, convertase)
Refseq ID (NCB1): NM_001223
SwissProt: P29466
Refseq protein ID (NCBI): NP_001214.1
SEQ ID No. 1
2060_11_at CASP1
>HG-U133_PLUS_2: 206011_AT
aatgctctaaaatccaacactgtgtgagcgcccacatgatattcaaaggaaatgtttattgaaacatttcaaattanagtttttggattagggatgctaaa
ccagtaagtatacagctgaattccaatatacaaaaatatctgaaatttgaaatacttctggtagcatgcattttggataagggataatcaagccatatacag
aaaatactgaagtaatgcctttcttctggtcagtgcagagcacgttgctcttctcccaaagtttttcatttagaccccttttgagattcatctgatattggcc
tagaagtggccgtaagactacaaaccctcacttttggatgtttctctccttcacctcagcagggtatatttaaacatagcatgtggtctttccttttaaaatt
gtgtatgttcccattggtggtataactttataacttgcatgtcttt
Genbank: AI719655 (nt 1539-1970)
SEQ ID No. 2
211367_s_at CASP1
>HG-U133_PLUS_2: 211367_S_AT
gagctgaggttgacatcacaggcatgacaatgctgctacaaaatctggggtacagcgtagatgtgaaaaaaaatctcactgcttcggacatgactaca
gagctggaggcatttgcacaccgcccagagcacaagacctctgacagcacgttcctggtgttcatgtctcatggtattcgggaaggcatttgtgggaa
gaaacactctgagcaagtcccagatatactacaactcaatgcaatctttaacatgttgaataccaagaactgcccaagtttgaaggacaaaccgaaggt
gatcatcatccaggcc
Genbank: U13699 (nt 275-561)
SEQ ID No. 3
211366_x_at CASP1
>HG-U133_PLUS_2: 211366_X_AT
gcacaagacctctgacagcacgttcctggtgttcatgtctcatggtattcgggaaggcatttgtgggaagaaacactctgagcaagtcccagatatact
acaactcaatgcaatctttaacatgttgaataccaagaactgcccaagtttgaaggacaaaccgaaggtgatcatcatccaggcctgccgtggtgaca
gccctggtgtggtgtggtttaaagattcagtaggagtttctggaaacctatctttaccaactacagaagagtttgaggatgatgctattaagaaagcccac
atagagaaggattttatcgctttctgctcttccacaccagataatgtttcttggagacatcccacaatgggctctgtttttattggaagactcattgaacata
tgcaagaatatgcctgttcctgtgatgtggaggaaattttccgcaaggttcgattttcatttgagcagccagatggtagagcgcagatgcccaccactgaa
agagtgactttgacaagatgtttctacctcttcccaggacattaaa
Genbank: U13698 (nt 402-925)
SEQ ID No. 4
209970_x_at CASP1
>HG-U133_PLUS_2: 209970_X_AT
cgaaggtgatcatcatccaggcctgccgtggtgacagccctggtgtggtgtggtttaaagattcagtaggagtttctggaaacctatctttaccaactac
agaagagtttgaggatgatgctattaagaaagcccacatagagaaggattttatcgctttctgctcttccacaccagataatgtttcttggagacatccca
caatgggctctgtttttattggaagactcattgaacatatgcaagaatatgcctgttcctgtgatgtggaggaaattttccgcaaggttcgattttcatttg
agcagccagatggtagagcgcagatgcccaccactgaaagagtgactttgacaagatgtttctacctcttcccaggacattaaa
Genbank: M87507 (nt 859-1133)
SEQ ID No. 5
211368_s_at CASP1
>HG-U133_PLUS_2: 211368_S_AT
aatgtttcttggagacatcccacaatgggctctgtttttattggaagactcattgaacatatgcaagaatatgcctgttcctgtgatgtggaggaaattttcc
gcaaggttcgattttcatttgagcagccagatggtagagcgcagatgcccaccactgaaagagtgactttgacaagatgtttctacctcttcccaggaca
ttaaaat
Genbank: U13700 (nt 73-258)
COP1 specific probes
caspase-1 dominant-negative inhibitor pseudo-ICE
Refseq ID (NCB1): NM_001017534
SwissProt: Q5EG05
Refseq protein ID (NCB1): NP_001017534.1
SEQ ID No. 6
1552703_s_at COP1
>HG-U133_PLUS_2: 1552703_S_AT
ttccatgggtgaaggtacaataaatggcttactggatgaattattacagacaagggtgctgaaccaggaagagatggagaaagtaaaacgtgaaaatg
ctacagttatggataagacccgagctttgattgactccgttattccgaaaggggcacaggcatgccaaatttgcatcacatacatttgtgaagaagacag
ttacctgg
Genbank: NM_052889 (nt 86-267)
SEQ ID No. 7
1552701 _a_at COP1
>HG-U133_PLUS_2: 1552701_A_AT
aggtccgatacctggaaattagcttagtacacaagactcccaattactattttct
Genbank: NM_052889 (nt 314-344)
PSME2 specific probe
proteasome (prosome, macropain) activator subunit 2 (PA28 beta)
Refseq ID (NCB1): NM_002818
SwissProt: Q2TNB3
Refseq protein ID (NCB1): NP_002809.2
SEQ ID No. 8
201762_s_at PSME2
>HG-U133_PLUS_2: 201762_S_AT
caacacctgatccccaagattgaagatggaaatgattttggggtagcaatccaggagaaggtgctggagagggtgaatgccgtcaagaccaaagtg
gaagctttccagacaaccatttccaagtacttctcagaacgtggggatgctgtggccaaggcctccaaggagactcatgtaatggattaccgggccttg
gtgcatgagcgagatgaggcagcctatggggagctcagggccatggtgctggacctgagggccttctatgctgagctttatcatatcatcagcagcaa
cc
Genbank: NM_002818 (nt 453-723)
F13A1 specific probe
coagulation factor XIII, A1 polypeptide
Refseq ID (NCB1): NM_000129
SwissProt: P00488
Refseq protein ID (NCB1): NP_000120.1
SEQ ID No. 9
203305_at F13A1
>HG-U133_PLUS_2: 203305_AT
gtccttcacatcaccattttgagacctcagcttggcactcaggtgctgaagggtaatatggactcagccttgcaaatagccagtgctagttctgacccaa
ccacagaggatgctgacatcatttgtattatgttccaaggctactacagagaaggctgcctgctatgtatttgcaaggctgatttatggtcagaatttccct
ctgatatgtctagggtgtgatttaggtcagtagactgtgattcttagcaaaaaatgaacagtgataagtatactgggggcaaaatcagaatggaatgctct
ggtctatataaccacatttctgagcctttgagactgttcctgagccttcagcactaacctatgagggtgagctggtcccctctatatatacatcatacttaac
tttactaagtaatctcacagcatttgccaagtctcccaatatccaatt
Genbank: NM_000129 (nt 3289-3718)
NCF4 specific probe
neutrophil cytosolic factor 4, 40 kDa
Refseq ID (NCB1): NM_000631
SwissProt: Q15080
Refseq protein ID (NCB1): NP_000622.2
SEQ ID No. 10
205147_x_at NCF4
>HG-U133_PLUS_2: 205147_X_AT
agcagaggctctatttgacttcactggaaacagcaaactggagctgaatttcaaagctggagatgtgatcttcctcctcagtcggatcaacaaagactg
gctggagggcactgtccggggagccacgggcatcttccctctctccttcgtgaagatcctcaaagacttccctgaggaggacgaccccaccaactgg
ctgcgttgctactactacgaagacaccatcagcaccatcaaggacatcgcggtggaggaagatctcagcagcactcccctattgaaagacctgctgg
agctcacaaggcgggagttccagagagaggacatagctctgaattaccgggacgctgagggggatctggttcggctgctgtcggatgaggacgtag
cgctcatggtgcggcaggctcgtggcctcccctcccagaagcgcctcttcccctggaagctgcacatcacgcagaaggacaactacagggtctaca
acacgatgccat
Genbank: NM_000631 (nt 690-1162)
M160 specific probe
Scavenger receptor cysteine-rich type 1 protein M160, precursor (CD163 molecule-like 1)
Refseq ID (NCB1): NM_174941
SwissProt: Q2M3B7
Refseq protein ID (NCB1): NP_777601.2
SEQ ID No. 11
223655_at M160 (CD163L1)
>HG-U133_PLUS_2: 223655_AT
tctatgggactgtcacgccaaaccctggggacagagtgactgtggacacaaggaagatgctggcgtgaggtgctctggacagtcgctgaaatcact
gaatgcctcctcaggtcgtttagcacttattttatccagtatctttgggctccttctcccggttctgtttattctatttctcacgtggtgccgagttcagaaa
caaaaacatctgcccctcagagtttcaaccagaaggaggggttctctcgaggagaatttattccatgagatggagacctgcctcaagagagaggacccac
atgggacaagaacctcagatgacacccccaaccatggttgtgaagatgctagcgacacatcgctgttgggagttcttcctgcctctgaagccacaaaa
tgactttagacttccagggctcaccagatcaacctctaaatat
Genbank: NM_174941 (nt 4000-4415)
CSF1R specific probe
colony stimulating factor 1 receptor
Refseq ID (NCB1): NM_005211
SwissProt: P07333
Refseq protein ID (NCB1): NP_005202.2
SEQ ID No. 12
203104_at CSF1R
>HG-U133_PLUS_2: 203104_AT
tgttggcctcgtgtttgctatgccaactagtagaaccttctttcctaatccccttatcttcatggaaatggactgactttatgcctatgaagtccccaggagc
tacactgatactgagaaaaccaggctctttggggctagacagactggcagagagtgagatctccctctctgagaggagcagcagatgctcacagacc
acactcagctcaggccccttggagcaggatggctcctctaagaatctcacaggacctcttagtctctgccctatacgccgccttcactccacagcctca
cccctcccacccccatactggtactgctgtaatgagccaagtggcagctaaaagttgggggtgttctgcccagtcccgtcattctgggctagaaggca
ggggaccttggcattggctggccacaccaagcaggaagcacaaactcccccaagctgactcatcctaactaacagtcacgccgtg
Genbank: NM_005211 (nt 3485-3942)
FCGR2A specific probe
Fc fragment of IgG, low affinity IIa, receptor (CD32)
Refseq ID (NCB1): NM_021642
SwissProt: P12318
Refseq protein ID (NCB1): NP_067674.2
SEQ ID No. 13
203561_at FCGR2A
>HG-U133_PLUS_2: 203561_AT
tgctgggatgaccagcatcagccccaatgtccagcctctttaacatcttctttcctatgccctctctgtggatccctactgctggtttctgccttctccatgc
tgagaacaaaatcacctattcactgcttatgcagtgccaagctccagaagaacaaagagcccaattaccagaaccacattaagtctccattgttttgcctt
gggatttgagaagagaattagagaggtgaggatctggtatttcctggactaaattccccttggggaagacgaagggatgctgcagttccaaaagagaa
ggactcttccagagtcatctacctgagtcccaaagctccctgtcctgaaagccacagacaatatggtcccaaatgactgactgcaccttctgtgcctca
gccgttcttgacatcaagaatcttctgttccacatccacacagccaatacaattagtcaaaccactgttattaacagatgtagcaacatgaaagacgctat
gttacaggttaca
Genbank: NM_021642 (nt 1710-2200)
KCNS3 specific probe
potassium voltage-gated channel, delayed-rectifier, subfamily S, member 3
Refseq ID (NCB1): NM_002252
SwissProt: Q9BQ31
Refseq protein ID (NCB1): NP_002243.3
SEQ ID No. 14
205968_at KCNS3
>HG-U133_PLUS_2: 205968_AT
attgtggtgagcgatcctgactccacagatgcttcaagcattgaagacaatgaggacatttgtaacaccacctccttggagaattgcacagcaaaatga
gcgggggtgtttgtgcctgtttctcttatcctttcccaacattaggttaacacagctttataaacctcagtgggttcgttaaaatcatttaattctcagggtg
tacctttccagccatagttggacattcattgctgaattctgaaatgatagaattgtctttatttttctctgtgaggtcaattaaatgccttgttctgaaattt
attttttacaagagagagttgtgatatagtttggaatataagataaatggtattgggtggggtttgtggctacagcttatgcatcattctgtgtttgtcattt
actcacattgagctaactttaaattactgacaagtagaatcaaaggtgcagctgactgagacgacatgc
Genbank: NM_002252 (nt 1521-1973)
IBS1 specific probe
DKFZP564O0823 protein
Refseq ID (NCB1): NM_015393
SwissProt: Q6UW12
Refseq protein ID (NCB1): NP_056208.2
SEQ ID No. 15
204687_at DKFZP564O0823 (IBS1)
>HG-U133_PLUS_2: 204687_AT
aatctctatttatctggttgtttctgacaggatgctgcctgcttggctctacaagctggaaagcagcttcttagctgcctaattaatgaaagatgaaaatagg
aagtgccctggagggggccagcaggtcacggggcagaatctctcaggttgctgtgggatctcagtgtgcccctacctgttctcccctccaggccacc
tgtctctgtaaaggatgtctgctctgttcaaaaggcagctgggatcccagcccacaagtgatcagcagagttgcatttccaaagaaaaaggctatgaga
tgagctgagttatagagagaaagggagaggcatgtacggtgtggggaagtggaagggaagctggcgggggagaaggaggctaacctgcactga
gtacttcattaggacaagtgagaatcagctattgataatggccagagatatccacagcttggaggagcccagagaccgtttgctttatacccacacagc
aactggtccactgctttactg
Genbank: NM_015393 (nt 1602-2089)
VSIG2 specific probe
V-set and immunoglobulin domain containing 2
Refseq ID (NCB1): NM_014312
SwissProt: Q96IQ7
Refseq protein ID (NCB1): NP_055127.2
SEQ ID No. 16
229369_at VSIG2
>HG-U133_PLUS_2: 229369_AT
ggggtggcgcaaggagggaggaaagggcttgagttaaaagcgggtgcctgcaaccctcaaactccgacatcattcagtgtgtttaggggcaggag
gtgttgttcagccgtggaatttgctggtggcagcagtgtaacctgtgtatttgagggtacaggcaancggtacagggtggagtggctggtccacaagct
gtggcagggaagctgtttgcaggactgccctgcc
Genbank: AI201858 (nt 940-1143)

Alternatively, the level of gene transcription is determined at protein level and the invention provides a method for identifying a candidate compound for the treatment of CVH, in particular for the treatment of IBS, said method comprising;
a) contacting a cell expressing at least one IBS-MSG with the compound to be tested;
b) determining the protein level of said IBS-MSG; and
c) comparing with the protein level of said IBS-MSG in the absence of said compound;

whereby a compound capable of opposing the change in protein level of the IBS-MSG observed in IBS, is identified as a candidate compound for the treatment of CVH, in particular for the treatment of IBS.

Preferably, the protein level is determined using an antibody that binds to an IBS-MSG product. In particular using an antibody which binds to a polypeptide encoded by an IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular from the group consisting of IBS1, COP1, PSME2, F13A1, NCF4, CSF1R, M160, KCNS3, VSIG2; more in particular from the group consisting of IBS1, PSME2, F13A1, NCF4, CSFR1 AND VSIG2; even more in particular from the group consisting of MUC20, VSIG2 and VSIG4; most particular the IBS-MSG used in the screening methods of the present invention consists of IBS1.

According to a particular embodiment, the methods of the invention comprise, in step (b) determining the determine the protein level of the gene product of at least two different genes, one of which is selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20 and one or more other genes is selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20, CASP1, FCGR2A and CKB; in particular determining the protein level of the gene product of at least two genes selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular at least two genes selected from the group consisting of IBS1, COP1, PSME2, F13A1, NCF4, CSF1R, M160, KCNS3, VSIG2; more in particular at least two genes from the group consisting of IBS1, PSME2, F13A1, NCF4, CSFR1 AND VSIG2; even more in particular at least two genes from the group consisting of MUC20, VSIG2 and VSIG4; further embodiments of the present invention comprise in step (b) determining the protein level of at least two gene products, one of which is IBS1-gene product, the other being selected from the group consisting of the gene-products of COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20, CASP1, FCGR2A and CKB.

Antibodies generated against polypeptides of the present invention may be obtained by administering the polypeptides or epitope-bearing fragments, analogs or cells expressing these to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975)256:495-497), the trioma technique, the human B-cell hybridoma technique (Kozbor et al., Immunology Today (1983)4:72) and the EBV-hybridoma technique (Cole et al., MONOCLONAL ANTIBODIES AND CANCER THERAPY, pp. 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography.

Antibodies against polypeptides of the present invention may also be employed to treat CVH, in particular for the treatment of IBS.

To determine the amount of protein, the antibodies according to the invention are used in conventional immunological techniques. Suitable immunological techniques are well known to those skilled in the art and include for example, ELISA, Western Blot analysis, competitive or sandwich immunoassays and the like. As is otherwise well known they all depend on the formation of an antigen-antibody immune complex wherein for the purpose of the assay, the antibody can be detectably labeled with, e.g. radio-, enzyme or fluorescent labels or it can be immobilized on insoluble carriers.

For example in an ELISA screening format the antibody is added to a solid phase (for example the bottom of a microplate) which is coated with either the protein or a peptide fragment thereof coupled to a carrier (such as BSA), and then, adding an anti-immunoglobin antibody (for example when the immunization is performed in mice, an anti-mouse immunoglobulin antibody is used, e.g. sheep-anti-mouse immunoglobulin (Ig)) conjugated with a detectable label such as an enzyme, preferably horseradish peroxidase, or a radioactive isotope such as ¹²⁵I.

In a preferred embodiment of the invention, the individual nucleic acid and/or gene product is initially used in a screening method to identify “candidate compounds” that bind specifically to the particular gene or gene product. Once identified, the candidate compound can be further used in cell-based or whole animal-based assays to determine its effect on expression of the particular nucleic acid, or expression or activity (i.e. function) of the gene product, relative to an untreated control cell or animal expressing the same nucleic acid and/or gene product. For the present invention, expression can also be detected in cells further treated or untreated with drugs commonly used to treat IBS, e.g. probiotics, including Lactobacillus and Bifidobacterium; anti-inflammatory agents, such as locally active 5-ASA compounds or corticosteroids, such as for example budenoside; mast cell stabilizers, PAR-2 antagonists and approaches that inhibit caspase activity, such as for example N¹-3-methylbutyryl-N⁴-6-aminohexanoyl-piperazine; CNI-1493 or pralnacasan. Cell culture assays using colon cells, e.g., Caco-2 or HT-29 cells, may be used to determine whether a test compound functions as a modulator of expression. In a specific embodiment, cells are contacted with a test compound and the effect of the compound on the expression is evaluated relative to a corresponding cell not contacted with a test compound. As used herein, the term “corresponding cell” refers to a cell in a separate sample from that of the test sample that is preferably of the same cell-type from the same tissue-type as the cell being tested.

It is accordingly an object of the present invention to provide a screening method to identify and obtaining a candidate compound for the treatment of CVH, in particular for the treatment of IBS, said method comprising;
a) incubating an IBS-MSG product with the compound to be tested; and
b) determining the capability of said compound to bind with the IBS-MSG product; wherein a compound capable of binding to the IBS-MSG product is a candidate compound for the treatment of IBS.

In these binding assays the IBS-MSG product typically consists of the polypeptide encoded by said gene or fragments thereof and the capability of the test compound to bind with said polypeptide is determined using art known procedures, such as for example described in Ausubel et al. (Current Protocols in Molecular Biology, Wiley Interscience, New York, 2001). In an alternative embodiment, the IBS-MSG product is a polynucleotide transcribed from the IBS-MSG gene or a fragment thereof.

In one particular working example, a candidate compound that binds to a polypeptide of the invention may be identified using a chromatography-based technique. For example, a recombinant polypeptide of the invention may be purified by standard techniques from cells engineered to express the polypeptide (e.g. those described above) and may be immobilized on a column. A solution of candidate compounds is then passed through the column, and a compound specific for the immobilized polypeptide of the invention is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds isolated by this method (or any other appropriate method) may, if desired, be further purified (e.g. by high performance liquid chromatography). In addition, these candidate compounds may be tested for their ability to alter (e.g. increase or decrease) the activity of a polypeptide of the invention. Compounds isolated by this approach may also be used, for example, as therapeutics to treat IBS in a human subject. Compounds that are identified as binding to a polypeptide of the invention with an affinity constant less than or equal to 10 μM are considered particularly useful in the invention and are hereinafter also referred to as specific binding agents.

Alternatively, the binding assay further comprises the presence of a specific binding agent for the IBS-MSG of interest, i.e. either an antibody or another agent known to bind with the gene of interest. For the IBS-MSGs of the present invention, a list of known commercially available antibodies and of known agonists is provided in the lists hereinafter. In the binding assay, the capability of the test compound to bind with the IBS-MSG is assessed by measuring the effect of the test compound on the interaction between the IBS-MSG and said specific binding agent.

### Examples of Commercially Available Antibodies (Monoclonal or Polyclonal) for Genes Listed in Table 1:

Anti-Human CASP1 Antibody
(Abnova Corporation, Calbiochem, Novus Biologicals)

Anti-Human NCF4 Antibody
(Abnova Corporation, Abcam, Genetex, Novus Biologicals)

Anti-Human Lysozyme Antibody
(BIODESIGN International)

Anti-Human PSME2 Antibody

(Abnova Corporation, Novus Biologicals)
Anti-Human HELLS Antibody

(Abnova Corporation, Bethyl Laboratories, Genetex, Novus Biologicals)

Anti-Human COP1 Antibody
(Abnova Corporation, IMGENEX, Novus Biologicals)

Anti-Human MCM5 Antibody
(Abcam, AbD Serotec, BD Biosciences Pharmingen, Bethyl Laboratories, BioLegend, GeneTex, Lab Vision, Novus Biologicals, Spring Bioscience)

Anti-Human TAP2 Antibody
(Abgent, BD Biosciences Pharmingen)

Anti VSIG2 Antibody
(Abcam: mouse monoclonal Cortical Thymocytes antibody, ab24235—reacts with human)

### Examples of Agonists for Proteins Encoded by the Genes Listed in Table 1:

LYSOZYME Activation (Agonists):
cyclosporin A (induction of lysozyme release)
1-ethyl-benzimidazolinone (1-EBIO)
Carbachol
Thapsigargin
Phenylephrine

NADPH OXIDASE Activation:
angiotensin II [Ang II]
PMA
TNF-alpha
growth factors
thrombin
phorbol myristate acetate (PMA)

PSME2 and TAP2 Activation:
interferon-gamma

For detection of molecules capable of binding to the genes of interest using the aforementioned screening assays, the molecule that specifically binds to the gene of interest (e.g. antibody, agonist or polynucleotide probe) can be detectably labeled by virtue of containing an atom (e.g. radionuclide), molecule (e.g. fluorescein), or complex that, due to a physical or chemical property, indicates the presence of the molecule. A molecule may also be detectably labeled when it is covalently bound to or otherwise associated with a “reporter” molecule (e.g. a biomolecule such as an enzyme) that acts on a substrate to produce a detectable atom, molecule or other complex. Detectable labels suitable for use in the present invention include any composition detectable by spectroscopic, photochemical, biochemical, immunochemical, electrical, optical or chemical means. Labels useful in the present invention include biotin for staining with labeled avidin or streptavidin conjugate, magnetic beads (e.g. Dynabeads'), fluorescent dyes (e.g. fluorescein, fluorescein-isothiocyanate (FITC), Texas red, rhodamine, green fluorescent protein, enhanced green fluorescent protein, lissamine, phycoerythrin, Cy2, Cy3, Cy3.5, Cy5, Cy5.5, Cy7, Fluor X [Amersham], SyBR Green I & II [Molecular Probes], and the like), radiolabels (e.g. ³H, ¹²⁵I, ³⁵S, ¹⁴C, or ³²P), enzymes (e.g. hydrolases, particularly phosphatases such as alkaline phosphatase, esterases and glycosidases, or oxidoreductases, particularly peroxidases such as horse radish peroxidase, and the like), substrates, cofactors, inhibitors, chemilluminescent groups, chromogenic agents, and colorimetric labels such as colloidal gold or colored glass or plastic (e.g. polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, chemilluminescent and radioactive labels may be detected using photographic film or scintillation counters, and fluorescent markers may be detected using a photodetector to detect emitted light (e.g. as in fluorescence-activated cell sorting). Enzymatic labels are typically detected by providing the enzyme with a substrate and detecting a colored reaction product produced by the action of the enzyme on the substrate. Colorimetric labels are detected by simply visualizing the colored label. Thus, for example, where the label is a radioactive label, means for detection include a scintillation counter, photographic film as in autoradiography, or storage phosphor imaging. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Also, simple colorimetric labels may be detected by observing the color associated with the label. Fluorescence resonance energy transfer has been adapted to detect binding of unlabeled ligands, which may be useful on arrays.

Evaluation of binding interactions may further be performed using Biacore technology, wherein the IBS-MSG polypeptide or its binding partner is bound to a micro chip, either directly by chemical modification or tethered via antibody-epitope association (e.g. antibody to the IBS-MSG polypeptide), antibody directed to an epitope tag (e.g. His tagged) or fusion protein (e.g. GST). A second protein or proteins is/are then applied via flow over the“chip” and the change in signal is detected. Finally, test compounds are applied via flow over the“chip” and the change in signal is detected.

Classes of compounds that may be identified by such screening assays include, but are not limited to, small molecules (e.g. organic or inorganic molecules which are less than about 2 kd in molecular weight, more preferably less than about 1 kd in molecular weight, and/or are able to cross the blood-brain barrier or gain entry into an appropriate cell and affect the expression of the relevant gene or the activity of the relevant gene product). Compounds identified by these screening assays may also include polypeptides, such as soluble peptides, fusion peptides, members of combinatorial libraries (such as those described by Lam et al., Nature 1991, 354:82-84; and by Houghten et al., Nature 1991, 354: 84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778); peptide libraries derived from the“phage method” (Scott and Smith, Science 1990, 249: 386-390; Cwirla, et al., Proc. Natl. Acad. Sci. USA 1990, 87:6378-6382; Devlin et al., Science 1990, 49:404-406); chemicals from other chemical libraries (Geysen et al., Molecular Immunology 1986, 23:709-715; Geysen et al., J. Immunologic Methods 1987, 102:259-274; Fodor et al., Science 1991, 251:767-773; Furka et al., 14th International Congress of Biochemistry 1988, Volume & num; 5, Abstract FR: 013; Furka, Int. J. Peptide Protein Res. 1991, 37:487-493; U.S. Pat. No. 4,631, 211; U.S. Pat. No. 5,010,175; Needels et al., Proc. Natl. Acad. Sci. USA 1993, 90:10700-4; Ohlmeyer et al., Proc. Natl. Acad. Sci. USA 1993, 90:10922-10926; PCT Publication No. WO 92/00252; and PCT Publication No. WO 94/28028); and large libraries of synthetic or natural compounds available from a variety of sources, including Maybridge Chemical Co. (Trevillet, Cornwall, UK), Comgenex (Princeton, N.J.), Brandon Associates (Merrimack, N.H.), Microsource (New Milford, Conn.), Aldrich (Milwaukee, Wis.), Pan Laboratories (Bothell, Wash.), and MycoSearch (NC) (see, e.g. Blondelle et al., TIBTech 1996, 14:60).

### Diagnostic Assays

This invention further relates to the use of polynucleotides of the present invention as diagnostic reagents. Detection of a mutated form of an IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular from the group consisting of IBS1, COP1, PSME2, F13A1, NCF4, CSF1R, M160, KCNS3, VSIG2; more in particular from the group consisting of IBS1, PSME2, F13A1, NCF4, CSFR1 AND VSIG2; even more in particular from the group consisting of MUC20, VSIG2 and VSIG4; most particular the IBS-MSG used in the diagnostic methods of the present invention consists of IBS1, will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

It will thus be appreciated that this invention provides a method of diagnosing a pathological condition or a susceptibility to a pathological condition in a subject related to CVH, in particular IBS, comprising:

(a) determining the presence or absence of a mutation in the polynucleotide according to the invention; and
(b) diagnosing a pathological condition or susceptibility to a pathological condition based on the presence or absence of said mutation.

The methods further include methods of determining whether or not a sample is indicative of IBS and/or a particular stage of IBS and/or indicative of a susceptibility of IBS based on step (a) described above.

Nucleic acids for diagnosis may be obtained from a subject's cells, such as from blood (including total blood, serum, plasma and in particular white blood cells), urine, saliva, fecal sample, fecal cells, tissue biopsy (in particular colon biopsy) or autopsy material. The genomic DNA may be used directly for detection or may be amplified enzymatically by using PCR or other amplification techniques prior to analysis. mRNA or cDNA may also be used in similar fashion. Deletions and insertions can be detected by a change in size of the amplified product in comparison to the normal genotype. Point mutations can be identified by hybridizing amplified DNA to labeled mammalian purine permease nucleotide sequences. Perfectly matched sequences can be distinguished from mismatched duplexes by RNase digestion or by differences in melting temperatures. DNA sequence differences may also be detected by alterations in electrophoretic mobility of DNA fragments in capilary electrophoresis columns or gels, with or without denaturing agents, or by direct DNA sequencing (e.g., Myers et al., Science (1985)230:1242). Sequence changes at specific locations may also be revealed by specific restriction endonucleases, nuclease protection assays, such as RNase and Si protection or a chemical cleavage method (see Cotton et al., Proc Natl Acad Sci USA (1985) 85: 4397-4401). In another embodiment, an array of oligonucleotides probes that specifically bind to the IBS-MSGs can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al., Science, Vol 274, pp 610-613 (1996)).

The diagnostic assays offer a process for diagnosing or determining a susceptibility to IBS through detection of mutations in the IBS-MSGs by the methods described. In addition, such disease may be diagnosed by methods comprising determining from a sample derived from a subject an abnormally decreased or increased level of polypeptide or mRNA, as well as by determining from said samples the presence of protein derivatives compared to the normal structure. Decreased or increased expression can be measured at the RNA level using any of the methods well known in the art for the quantitation of polynucleotides, such as, for example; nucleic acid amplification, for instance via PCR, RT-PCR; RNase protection; Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays. Assay techniques that can be used to determine the presence of protein derivatives or variants comprise amongst others mass spectrometry.

Thus in another aspect, the present invention provides a method for detecting and/or monitoring IBS in a subject, said method comprising:

(a) determining, in a biological sample of said subject, the level of gene transcription of an IBS-MSG;
(b) comparing the level of gene transcription with the level of gene transcription in a normal control sample; and
(c) producing a diagnosis based on the result from step b).

The methods further include methods of determining whether or not a sample is indicative of IBS and/or a particular stage of IBS and/or indicative of a susceptibility of IBS based on steps (a) and (b) described above.

The IBS-MSG as used in the diagnostic methods of the present invention is typically selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular from the group consisting of IBS1, COP1, PSME2, F13A1, NCF4, CSF1R, M160, KCNS3, VSIG2; more in particular from the group consisting of IBS1, PSME2, F13A1, NCF4, CSFR1 and VSIG2; even more in particular from the group consisting of MUC20, VSIG2 and VSIG4.

It is accordingly an object of the present invention to provide a method for detecting and/or monitoring IBS in a subject, said method comprising:

(a) determining, in a biological sample of said subject, the level of gene transcription of an IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular from the group consisting of IBS1, COP1, PSME2, F13A1, NCF4, CSF1R, M160, KCNS3 and VSIG2; more in particular from the group consisting of IBS1, PSME2, F13A1, NCF4, CSFR1 and VSIG2; even more in particular from the group consisting of MUC20, VSIG2 and VSIG4;
(b) comparing the level of gene transcription with the level of gene transcription in a normal control sample; and
(c) producing a diagnosis and/or determining whether or not the sample is indicative of (a particular type of) IBS based on the result from step b).

In a further embodiment of the method for detecting and/or monitoring IBS in a subject, step a) includes determining two, three, four, five, six, seven, eight or more of the IBS-MSGs listed above.

In a particular embodiment, the biological sample of said patient is a sample of colon tissue, more particularly, a sample of mucosal colon tissue.

According to a particular embodiment, the methods for detecting and/or monitoring IBS in a subject according to the present invention comprise, in step (a) determining the expression of at least two different genes, one of which is selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20 and one or more other genes is selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20, CASP1, FCGR2A and CKB; in particular determining the expression of at least two genes selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular at least two genes selected from the group consisting of IBS1, COP1, PSME2, F13A1, NCF4, CSF1R, M160, KCNS3, VSIG2; more in particular at least two genes from the group consisting of IBS1, PSME2, F13A1, NCF4, CSFR1 AND VSIG2; even more in particular at least two genes from the group consisting of MUC20, VSIG2 and VSIG4; further embodiments of the present invention comprise in step (a) determining the expression of at least two genes, one of which is IBS1, the other being selected from the group consisting of COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20, CASP1, FCGR2A and CKB.

For each of the embodiments described thereof, step (b) consists of comparing the expression level of said at least two genes with the level of transcription of said genes in a healthy control sample.

In particular it consists of determining the expression levels of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; more in particular of the expression levels of IBS1, COP1, PSME2, F13A1, NCF4, CSF1R, M160, KCNS3, VSIG2; even more in particular of the expression levels of IBS1, PSME2, F13A1, NCF4, CSFR1 and VSIG2; most in particular of the expression levels of MUC20, VSIG2 and VSIG4.

In any of the aforementioned methods for detecting and/or monitoring IBS in a subject, the use of the IBS-MSGs as identified in the present application may be combined with other genes such as for example CASP1, FCGR2A, SLC6A4, SLC12A2, SCNN1A, OPRIM, AQP3, NKCC1, THP1, CCL2/MCP-1, CXCL8/IL-8, IL-10, GNB3, ADRA2A, TNFα, CCK1, IL-4, IL-4R, IL-6, IL-7, IL-1B and CKB.

It is accordingly an object of the present invention to provide a method for detecting and/or monitoring IBS in a subject wherein step a) according to any of the aforementioned embodiments further includes determining the level of gene transcription of at least one, two, three or more genes known as an IBS marker, in particular selected from the group consisting of CASP1, FCGR2A and CKB.

In order to detect IBS in a subject one would have to compare the expression levels of the IBS-MSGs in a sample of said subject with a normal control sample. Changes in the expression levels of the IBS-MSGs in the sample of said subject compared to the expression levels of said genes in the control sample are indicative for a diagnosis of, or susceptibility to IBS in said subject. For example, if the level of any of the following IBS-MSGs: IBS1, VSIG2 or MUC20 is increased (e.g. 30%, 40%, 50%, 60%, 70%, 80%, 90%, 100%, 110%, 120%, 130%, 140%, 150% or more), relative to the control sample, this is considered a positive indicator for IBS in said subject. In another example, if the level of any of the following IBS-MSGs: COP1, PSME2, F13A1, NCF4, CSF1R, M160, KCNS3, LYZ, MS4A4A, HELLS, RFC4, MCM5, TAP2, LRAP, DTL or VSIG4 is decreased (e.g. 30%, 40%, 50%, 60%, 70%, 80%, 90% or more), relative to the control sample, this is considered a positive indicator for IBS in said subject. Additionally or alternatively, the differences in expression can be expressed as ‘fold-changes’ compared to the expression level observed in control samples. In such embodiments, a 1,4 fold change will correspond to an increase of 40%, etc. Generally, a decrease in expression will be referred to as 0.6 fold change for a decrease of 40%. ***PLEASE CHECK***

It is accordingly an object of the present invention to provide a method for identifying IBS in a subject, said method comprising;

(a) determining, in a biological sample of said subject, the level of gene transcription of an IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular from the group consisting of IBS1, COP1, PSME2, F13A1, NCF4, CSF1R, M160, KCNS3 and VSIG2; more in particular from the group consisting of IBS1, PSME2, F13A1, NCF4, CSFR1 and VSIG2; even more in particular from the group consisting of MUC20, VSIG2 and VSIG4; and
(b) comparing the level of gene transcription with the level of gene transcription in a normal control sample;
wherein an increase in the level of gene transcription of a gene selected from the group consisting of IBS1, VSIG2 and MUC20 or a decrease in the level of gene transcription of a gene selected from the group consisting of COP1, PSME2, F13A1, NCF4, CSF1R, M160, KCNS3, LYZ, MS4A4A, HELLS, RFC4, MCM5, TAP2, LRAP, DTL and VSIG4, is an indication of IBS in said subject.

As described hereinbefore, the level of gene transcription is determined either at the protein level, preferably using antibodies that bind to the IBS-MSG polypeptide, or at the gene transcription level, preferably using probes that specifically bind to an oligonucleotide transcribed from said IBS-MSG, preferably at the cDNA or mRNA level. In a particular embodiment the level of gene transcription is determined using array technology, either at the oligonucleotide level using specific probes as described hereinbefore or at the protein level using specific binding agents, preferably antibodies as described hereinafter.

Hence, in a further embodiment the present invention, the level of gene expression in the aforementioned methods is assessed using a probe that specifically binds to cDNA or mRNA of the gene of interest; in particular using microarray technology. It is accordingly an object of the present invention to provide a method for determining and monitoring IBS in a subject, wherein the level of gene transcription is assessed using an array of oligonucleotide probes that bind to the IBS-MSGs; in one embodiment said genes are selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular from the group consisting of IBS1, COP1, PSME2, F13A1, NCF4, CSF1R, M160, KCNS3 and VSIG2; more in particular from the group consisting of IBS1, PSME2, F13A1, NCF4, CSFR1 and VSIG2; even more in particular from the group consisting of MUC20, VSIG2 and VSIG4. As already mentioned hereinbefore, the arrays of oligonucleotide probes for the IBS-MSGs are optionally combined with probes that specifically bind to other genes, in particular selected from the group consisting of CASP1, FCGR2A and CKB. Accordingly, in a further object of the present invention the expression levels of the genes are determined using an array of the probes enlisted in Table 1, more in particular using an array of the probes enlisted in Table 2.

Protein arrays are typically solid-phase, ligand binding assay systems using immobilized proteins on surfaces which include glass, membranes, microtiter wells, mass spectrometer plates and beads or other particles. Automated multi-well formats are the best developed and automated 96-well plate-based screening systems are the most widely used. For a description of protein arrays that can be used in the methods of the presents invention see U.S. Pat. Nos. 6,475,809; 6,406,921 and 6,197,599; and PCT publications WO 00/04389 and WO 00/07024.

For construction of arrays, sources of proteins include cell-based expression systems for recombinant proteins, purification from natural sources, production in vitro by cell-free translation systems, and synthetic methods for peptides. For capture arrays and protein function analysis, it is important that proteins should be correctly folded and functional; this is not always the case, e.g. where recombinant proteins are extracted from bacteria under denaturing conditions, whereas other methods (isolation of natural proteins, cell free synthesis) generally retain functionality. However, arrays of denatured proteins are useful in screening antibodies for cross-reactivity, identifying auto-antibodies and selecting ligand binding proteins.

The immobilization method used should be reproducible, applicable to proteins of different properties (size, hydrophilic, hydrophobic), amenable to high throughput and automation, and compatible with retention of fully functional protein activity. Both covalent and noncovalent methods of protein immobilization are used. Substrates for covalent attachment include glass slides coated with amino-or aldehyde-containing silane reagents (Telechem). In theVersalinx' system (Prolinx), reversible covalent coupling is achieved by interaction between the protein derivatized with phenyldiboronic acid, and salicylhydroxamic acid immobilized on the support surface. Covalent coupling methods providing a stable linkage can be applied to a range of proteins. Noncovalent binding of unmodified protein occurs within porous structures such as HydroGel (PerkinElmer), based on a 3-dimensional polyacrylamide gel.

Thus, in a further embodiment the present invention provides a method for identifying and/or monitoring IBS in a subject said method comprising;

a) determining, in a biological sample of said subject, the protein level of at least one IBS-MSG protein;
(b) comparing the protein level with the protein level in a normal control sample; and
(c) producing a diagnosis based on the result from step b).

Preferably, the protein level is determined using at least one antibody that binds to an IBS-MSG protein. In particular using one or more antibodies, each of which binds to a polypeptide encoded by an IBS-MSG selected from IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular from the group consisting of IBS1, COP1, PSME2, F13A1, NCF4, CSF1R, M160, KCNS3, VSIG2; more in particular from the group consisting of IBS1, PSME2, F13A1, NCF4, CSFR1 AND VSIG2; even more in particular from the group consisting of MUC20, VSIG2 and VSIG4; in a most particular embodiment the protein level is determined using an antibody specific for IBS1.

In an alternative embodiment the method is not limited to at least one protein according to the invention, but requires the simultaneous assessment of the expression levels of the group of proteins identified as being involved in IBS, i.e. the proteins encoded by the IBS-MSG enlisted in Table 1, in one embodiment consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular from the group consisting of IBS1, COP1, PSME2, F13A1, NCF4, CSF1R, M160, KCNS3, and VSIG2; more in particular from the group consisting of IBS1, PSME2, F13A1, NCF4, CSFR1 and VSIG2; even more in particular from the group consisting of MUC20, VSIG2 and VSIG4. In a preferred embodiment the simultaneous assessment of the expression levels of the group of proteins is done using array technology, in particular using immunological methods (such as ELISAs and RIAs). As mentioned hereinbefore, in protein arrays the primary agent, typically an antibody or protein that recognizes the IBS proteins is bound to a solid support (e.g. a membrane or a microtiter plate). Using this solid support the IBS proteins can be extracted from the biological sample and quantified using a secondary agent (e.g. a second antibody recognizing a second epitope in the IBS protein or an antibody or protein that recognizes the primary antibody) conjugated with a detectable label such as an enzyme, preferably horseradisch peroxidase, or a reactive isotope such as ¹²⁵I.

It is thus an object of the present invention to provide a method for detecting and/or monitoring IBS in a subject, said method comprising;
a) contacting a biological sample of said subject with at least one agent that specifically binds with an IBS-MSG polypeptide;
b) determining the level of binding of the agent to the polypeptide;
c) comparing the level of binding of the agent in said biological sample with the level of binding of the agent in a normal control sample; and
d) producing a diagnosis or determining whether or not the sample is indicative of IBS and/or a particular status of IBS based on the result of step c).

As already mentioned hereinbefore, in a preferred embodiment the assay is performed using a protein array of IBS-MSG polypeptide specific antibodies; in one embodiment the two or more specific antibodies are each reactive with polypeptides of IBS-MSGs selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular reactive with polypeptides of IBS-MSGs selected from the group consisting of IBS1, COP1, PSME2, F13A1, NCF4, CSF1R, M160, KCNS3 and VSIG2; more in particular reactive with polypeptides of IBS-MSGs selected from the group consisting of IBS1, PSME2, F13A1, NCF4, CSFR1 and VSIG2; even more in particular reactive with IBS-MSGs selected from the group consisting of MUC20, VSIG2 and VSIG4. As already mentioned hereinbefore, the protein arrays for the IBS-MSGs are optionally combined with agents that specifically bind to other genes, in particular selected from the group consisting of CASP1, FCGR2A and CKB.

In order to detect IBS in a subject one would have to compare the level of binding of the agent to the IBS-MSG polypeptides in a sample of said subject with the level of binding in a normal control sample. Changes in the binding levels are indicative for a diagnosis of, or susceptibility to IBS in said subject. For example, if the binding level of any of the following IBS-MSG polypeptides: IBS1, VSIG2 or MUC20 is increased (e.g. 30%, 40%, 50%, 60%, 70%, 80%, 90%, 100%, 110%, 120%, 130%, 140%, 150% or more), relative to the control sample, this is considered a positive indicator for IBS in said subject. In another example, if the binding level of any of the following IBS-MSG polypeptides: COP1, PSME2, F13A1, NCF4, CSF1R, M160, KCNS3, LYZ, MS4A4A, HELLS, RFC4, MCM5, TAP2, LRAP, DTL or VSIG4 is decreased (e.g. 30%, 40%, 50%, 60%, 70%, 80%, 90% or more), relative to the control sample, this is considered a positive indicator for IBS in said subject The diagnostic methods described herein can also be used to monitor the IBS in a subject or to determine the dosages of therapeutic compounds. In one example, a therapeutic compound is administered and the level of expression of an IBS-MSG is determined during the course of therapy.

Therapeutics that modulate the expression of any one or more of the IBS-MSGs are considered particularly useful in the invention. In one example, a therapeutic agent that decreases, by 10%, 20%, 30%, 40%, 50%, 60%, 70%, 80%, 90%, 100%, 110%, 120%, 130%, 140%, 150% or more, the expression level of any of the following IBS-MSGs: IBS1, VSIG2 or MUC20 during the course of therapy, is considered to be an effective therapeutic agent or an effective dosage of a therapeutic agent. In another example, a therapeutic agent that increases, by 10%, 20%, 30%, 40%, 50%, 60%, 70%, 80%, 90% or more the expression level of any of the following IBS-MSGs: COP1, PSME2, F13A1, NCF4, CSF1R, M160, KCNS3, LYZ, MS4A4A, HELLS, RFC4, MCM5, TAP2, LRAP, DTL or VSIG4 during the course of therapy, is considered to be an effective therapeutic agent or an effective dosage of a therapeutic agent.

### Diagnostic Kits

The invention also encompases kits for detecting the presence of an IBS-MSG product in a biological sample, the kit comprising the components required to carry out any of the diagnostic assays described above and instructions for the use of the components for assessing expression of the IBS-MSGs in a biological sample and diagnosing IBS in a subject.

It is accordingly an object of the present invention to provide a diagnostic kit which comprises:

(a) at least one probe that specifically binds to an IBS-MSG; in particular to an IBS-MSG selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; more in particular with an IBS-MSGs selected from the group consisting of IBS1, COP1, PSME2, F13A1, NCF4, CSF1R, M160, KCNS3 and VSIG2; even more in particular with an IBS-MSGs selected from the group consisting of IBS1, PSME2, F13A1, NCF4, CSFR1 and VSIG2; most particular with an IBS-MSGs selected from the group consisting of MUC20, VSIG2 and VSIG4; or
(b) at least one agent that specifically binds to an IBS-MSG polypeptide or a fragment thereof; in particular to an IBS-MSG polypeptide selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; more in particular with an IBS-MSG polypeptide selected from the group consisting of IBS1, COP1, PSME2, F13A1, NCF4, CSF1R, M160, KCNS3 and VSIG2; even more in particular with an IBS-MSG polypeptide selected from the group consisting of IBS1, PSME2, F13A1, NCF4, CSFR1 and VSIG2; most particular with an IBS-MSG polypeptide selected from the group consisting of MUC20, VSIG2 and VSIG4. According to a particular embodiment, the kits of the invention comprise two or more probes and/or binding agents for determining the expression of at least two different genes, one of which is selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20 and one or more other genes is selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20, CASP1, FCGR2A and CKB; in particular two or more probes or binding agents for determining the expression of at least two genes selected from the group consisting of IBS1, COP1, PSME2, F13A1, NCF4, CSFR1, M160, KCNS3, LYZ, MS4A4A, HELLS, FRC4, MCM5, TAP2, LRAP, DTL, VSIG2, VSIG4 and MUC20; in particular two or more probes for determining the expression at least two genes selected from the group consisting of IBS1, COP1, PSME2, F13A1, NCF4, CSF1R, M160, KCNS3, VSIG2; more in particular at least two probes and/or binding agents for determining the expression of two or more genes from the group consisting of IBS1, PSME2, F13A1, NCF4, CSFR1 AND VSIG2; even more in particular for determining the expression of at least two genes from the group consisting of MUC20, VSIG2 and VSIG4; further embodiments of the kits of the present invention comprise two or more probes and/or binding agents for determining the expression of at least two genes, one of which is IBS1, the other being selected from the group consisting of COP1, PSME2, F13A1, NCF4, CSFR1, M160, KCNS3, LYZ, MS4A4A, HELLS, FRC4, MCM5, TAP2, LRAP, DTL, VSIG2, VSIG4, MUC20, CASP1, FCGR2A and CKB.

It will be appreciated that in any such kit, (a) or (b) may comprise additional components required to carry out any of the diagnostic assays described hereinbefore. For example, in a preferred embodiment the agent that specifically binds to an IBS-MSG polypeptide would be an antibody, and the kit would further include components required to quantificate binding between the antibodies and the IBS-MSG polypeptides. In one embodiment of the invention, such an immunological kit includes a solid support (e.g. a membrane or a microtiter plate) coated with a primary agent (e.g. an antibody or protein that recognizes the antigen), standard solutions of purified protein for preparation of a standard curve, a body fluid (e.g. serum or urine) control for quality testing of the analytical run, a secondary agent (e.g. a second antibody reactive with a second epitope in the antigen to be detected or an antibody or protein that recognizes the primary antibody) conjugated to a label or an enzyme such as horse radish peroxidase or otherwise labeled, a substrate solution, a stopping solution, a washing buffer and an instruction manual. The membrane can be supported on a dipstick structure where the sample is deposited on the membrane by placing the dipstick structure into the sample or the membrane can be supported in a lateral flow cassette where the sample is deposited on the membrane through an opening in the cassette. The kit can also be in an array format and can include an array of polypeptides of the invention or binding molecules that specifically bind polypeptides of the invention arranged on a biochip, such as, for example, a GeneChip™.

The diagnostic kits also generally include a label or instructions for the intended use of the kit components and a reference sample or purified proteins to be used to establish a standard curve. In one example, the kit contains instructions for the use of the kit for the diagnosis of IBS. In yet another example, the kit contains instructions for the use of the kit to monitor therapeutic treatment or dosage regimens for the treatment of IBS. It will be understood that the reference sample values will depend on the intended use of the kit. For example, the sample can be compared to a normal reference value, wherein an alteration in the levels of one or more of the polypeptides of the invention or a metric using levels of one or more of the polypeptides of the invention is indicative of IBS, or a predisposition to IBS. In another example, a kit used for therapeutic monitoring can have a reference value that is indicative of IBS, wherein an alteration in the level of one or more of the polypeptides of the invention or a metric using levels of one or more of the polypeptides of the invention relative to the reference sample can be used to indicate therapeutic efficacy or effective dosages of therapeutic compounds.

### Therapeutic Utility

The present invention features methods and compositions for treating or preventing CVH, in particular for treating or preventing IBS in a subject. It has been discovered that levels of IBS1, VSIG2 and MUC20 are increased in subjects having IBS. Therefore, the invention includes methods and agents that decrease the expression levels or biological activity of any one or more of these polypeptides or nucleic acid molecules. Such agents which are described in more detail below, include compounds that down-regulate or inhibit the biological activity of any one or more of the above polypeptides; immunological/vaccine formulations; a purified antibody or antigen-binding fragment that specifically binds any one of the above polypeptides; antisense nucleobase oligomers; and dsRNAs targeting any of the above polypeptides.

In a first aspect, reduction of the biological activity of the polypeptides that are upregulated in IBS will be established using a pharmaceutical composition comprising a therapeutically effective amount of an antagonist, e.g. peptide or small molecule compound, in combination with a pharmaceutically acceptable carrier or excipient.

A further aspect of the invention relates to an immunological/vaccine formulation (composition) which, when introduced into a mammalian host, induces an immunological response in that mammal to a polypeptide of the present invention wherein the composition comprises a polypeptide or polynucleotide of the present invention. The vaccine formulation may further comprise a suitable carrier. Since a polypeptide may be broken down in the stomach, it is preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

In still another approach, expression of the upregulated genes can be inhibited using expression blocking techniques. Known such techniques involve the use of antisense sequences, either internally generated or externally administered (see, for example, O'Connor, J. Neurochem (1991) 56:560; Oligodeoxynucleotides as Antisense Inhibitors of Gene Expression, CRC Press, Boca Raton, Fla. (1988)). Alternatively, oligonucleotides which form triple helices (“triplexes”) with the gene can be supplied (see, for example, Lee et al., Nucleic Acids Res (1979)6:3073; Cooney et al., Science (1988)241:456; Dervan et al., Science (1991)251:1360). These oligomers can be administered per se or the relevant oligomers can be expressed in vivo. Synthetic antisense or triplex oligonucleotides may comprise modified bases or modified backbones. Examples of the latter include methylphosphonate, phosphorothioate or peptide nucleic acid backbones. Such backbones are incorporated in the antisense or triplex oligonucleotide in order to provide protection from degradation by nucleases and are well known in the art. Antisense and triplex molecules synthesised with these and/or other modified backbones also form part of the present invention.

In another process for inhibiting expression of a target gene in a cell, RNA with partial or fully double-stranded character is introduced into the cell or into the extracellular environment. Inhibition is specific in that a nucleotide sequence from a portion of the target gene is chosen to produce inhibitory RNA. The RNA may comprise one or more strands of polymerized ribonucleotide; it may include modifications to either the phosphate-sugar backbone or the nucleoside. The double-stranded structure may be formed by a single self-complementary RNA strand or two complementary strands. Inhibition is sequence-specific in that the nucleotide sequences corresponding to the duplex region of the RNA are targeted for genetic inhibition. RNA containing a nucleotide sequence identical to a portion of the target sequence is preferred. Examples of RNA inhibition technology can be found in International Patent Application WO 99/32619.

In addition, expression of the upregulated IBS-MSG proteins may be prevented by using ribozymes specific to the mRNA sequence encoding said protein. Ribozymes are catalytically active RNAs that can be natural or synthetic (see for example Usman, N, et al., Curr. Opin. Struct. Biol (1996)6(4), 527-33.) Synthetic ribozymes can be designed to specifically cleave the aforementioned mRNAs at selected positions thereby preventing translation of said mRNAs into functional polypeptide. Ribozymes may be synthesised with a natural ribose phosphate backbone and natural bases, as normally found in RNA molecules. Alternatively the ribozymes may be synthesised with non-natural backbones to provide protection from ribonuclease degradation, for example, 2′-O-methyl RNA, and may contain modified bases.

As another alternative, antibodies that bind to and neutralize the activity of the upregulated IBS-MSGs mentioned above, can be used to prevent or treat IBS in a subject. Antibodies generated against polypeptides of the present invention may be obtained by administering the polypeptides or epitope-bearing fragments, analogs or cells expressing these to an animal, preferably a non-human animal, using routine protocols. Antibodies can be polyclonal or monoclonal; monoclonal antibodies are preferred. For preparation of monoclonal antibodies, any technique, which provides antibodies produced by continuous cell line cultures, can be used. Examples include the hybridoma technique (Kohler, G. and Milstein, C., Nature (1975)256:495-497), the trioma technique, the human B-cell hybridoma technique (Kozbor et al., Immunology Today (1983)4:72) and the EBV-hybridoma technique (Cole et al., MONOCLONAL ANTIBODIES AND CANCER THERAPY, pp. 77-96, Alan R. Liss, Inc., 1985). Monoclonal antibodies, particularly those derived from rodents including mice, have been used for treatment of various diseases; however, there are limitations to their use including the induction of a human anti-mouse immunoglobulin response that causes rapid clearance and a reduction in the efficacy of the treatment. For example, a major limitation in the clinical use of rodent monoclonal antibodies is an anti-globulin response during therapy (Miller et al., Blood, 62:988-995 1983; Schroff et al., Cancer Res., 45:879-885, 1985).

The art has attempted to overcome this problem by constructing “chimeric” antibodies in which an animal antigen-binding variable domain is coupled to a human constant domain (U.S. Pat. No. 4,816,567; Morrison et al., Proc. Natl. Acad. Sci. USA, 81:6851-6855, 1984; Boulianne et al., Nature, 312:643-646, 1984; Neuberger et al., Nature, 314:268-270, 1985). Chimerized antibodies preferably have constant regions derived substantially or exclusively from human antibody constant regions and variable regions derived substantially or exclusively from the sequence of the variable region from a mammal other than a human. Such humanized antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Methods for humanizing non-human antibodies are well known in the art (for reviews see Vaswani and Hamilton, Ann Allergy Asthma Immunol., 81:105-119, 1998 and Carter, Nature Reviews Cancer, 1:118-129, 2001). Generally, a humanized antibody has one or more amino acid residues introduced into it from a source that is non-human. These non-human amino acid residues are often referred to as import residues, which are typically taken from an import variable domain. Humanization can be essentially performed following the methods known in the art (Jones et al., Nature, 321:522-525, 1986; Riechmann et al., Nature, 332:323-329, 1988; and Verhoeyen et al., Science, 239:1534-1536 1988), by substituting rodent CDRs or other CDR sequences for the corresponding sequences of a human antibody. Accordingly, such humanized antibodies are chimeric antibodies wherein substantially less than an intact human variable domain has been substituted by the corresponding sequence from a non-human species (see for example, U.S. Pat. No. 4,816,567). In practice, humanized antibodies are typically human antibodies in which some CDR residues and possibly some FR residues are substituted by residues from analogous sites in rodent antibodies (Presta, Curr. Op. Struct. Biol., 2:593-596, 1992).

A cocktail of the monoclonal antibodies of the present invention can be used as an effective treatment for pregnancy related hypertensive disorders, such as pre-eclampsia or eclampsia. The cocktail may include as few as two, three, or four different antibodies or as many as six, eight, or ten different antibodies. In addition, the antibodies of the present invention can be combined with drugs currently used to treat IBS, e.g., CNI-1493 or any other medication used to treat IBS, or the symptoms associated with IBS.

Non-limiting examples of antibodies that are useful in the methods of the invention are as follows: anti-IBS1; anti-VSIG2, including the commercially available anti-VSIG2 antibody from Abcam [ab24235 a mouse monoclonal Cortical Thymocytes antibody] and anti-MUC20.

It has also been found that the levels of COP1, PSME2, F13A1, NCF4, CSF1R, M160, KCNS3, LYZ, MS4A4A, HELLS, RFC4, MCM5, TAP2, LRAP, DTL and VSIG4 are decreased in subjects having IBS. Therefore, the invention also includes any methods and agents that increase the expression levels or biological activity of any one or more of these polypeptides or nucleic acid molecules. Such agents which are described in more detail below, include compounds that upregulate or increase the biological activity of any one or more of the polypeptides, including the oligonucletides encoding these polypeptides or purified forms of the polypeptides themselves.

For treating abnormal conditions related to an under-expression of proteins, several approaches are also available. One approach comprises administering to a subject a therapeutically effective amount of a compound which activates a polypeptide of the present invention, i.e., an agonist as described above, in combination with a pharmaceutically acceptable carrier, to thereby alleviate the abnormal condition. Examples of IBS-MSG agonists useful in a method according to the invention are; Lysozyme (LYZ) agonists such as for example cyclosporin A (induction of lysozyme release), 1-ethyl-benzimidazolinone (1-EBIO), Carbachol, Thapsigargin and Phenylephrine; activators of NADPH oxidase such as for example angiotensin II [Ang II], PMA, TNF-, growth factors, thrombin, and phorbol myristate acetate (PMA); and induction of PSME2 and TAP2 by interferon-gamma.

Alternatively, gene therapy may be employed to effect the endogenous production of mammalian purine permease by the relevant cells in the subject. For example, a polynucleotide of the invention may be engineered for expression in a replication-defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For an overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996). Another approach is to administer a therapeutic amount of a polypeptide of the present invention in combination with a suitable pharmaceutical carrier.

Based on the above, in a further aspect, the present invention provides for pharmaceutical compositions comprising a therapeutically effective amount of a polypeptide, such as the soluble form of a polypeptide of the present invention, agonist/antagonist peptide or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. Polypeptides and other compounds of the present invention may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a polypeptide or other compounds of the present invention can be formulated in an enteric or an encapsulated formulation, oral administration may also be possible. Administration of these compounds may also be topical and/or localized, in the form of patches, salves, pastes, gels, and the like.

The dosage range required depends on the choice of peptide or other compounds of the present invention, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### Experimental Procedures

It has been an objective of the present invention to further understand the molecular mechanisms in the pathogenesis of IBS, and performed a microarray expression profiling study of mucosa of the sigmoid colon using biopsies that were collected in the same manner as in routine clinical practice.

As explained in more detail hereinafter, our study included 36 IBS patients (21 IBS-D and 15 IBS-C) and 25 healthy control subjects. All patients fulfilled the Rome II criteria for IBS diagnosis (Thompson et al., 1999) and underwent a thorough clinical examination to exclude other gastrointestinal disorders. Patients were selected based on their predominant bowel dysfunction which was confirmed at the time of the study by means of a standard questionnaire. Two sigmoid colon biopsies were collected from each participant during a sigmoidoscopy examination. An additional third colon biopsy was collected 2-3 months later from 10 subjects (5 IBS patients and 5 healthy controls) in order to assess the stability of the molecular signatures in health and IBS.

### Methods

### Recruitment of Subjects and Collection of Colon Biopsy Samples

IBS participants were selected from an administrative database of 752 patients with IBS residing within 150 miles radius of Rochester (Minnesota, U.S.A.), and were recruited by mailing. All IBS patients had already been evaluated by a staff gastroenterologist by clinically indicated tests including endoscopy, biopsies and tests of rectal evacuation. Patients were selected based on their predominant bowel dysfunction which was confirmed at the time of study by means of a standard questionnaire (Talley et al., 1990). Healthy volunteers were recruited by public advertisement in Rochester, Minn. All participants who responded to a letter inviting their participation signed informed consent for the study, which was approved by the Mayo Clinic Institutional Review Board. Every participant completed a validated bowel disease questionnaire (Talley et al., 1990) including questions to correspond to Rome II criteria (Thompson et al., 1999). The bowel disease questionnaire also includes a psychosomatic symptom checklist intended to identify somatization disorders and symptoms to characterize non-ulcer dyspepsia, and has been used extensively in epidemiological studies.

Participants attended the General Clinical Research Center in Charlton 7 to undergo a flexible sigmoidoscopy for the collection of colonic biopsies. Due to the risk of bleeding from the biopsy procedure, participants taking aspirin or anticoagulants were excluded if these medications could not be stopped at least 1 week prior to the endoscopy. Two phosphosodau enemas (Fleet® enema, C. B Fleet, Lynchburg, Va.) were administered one hour prior to sigmoidoscopy. Biopsies were taken from normal appearing mucosa only, i.e., areas with edema due to endoscope pressure were avoided. The sigmoidoscopy was performed without sedation as is the clinical standard at Mayo Clinic, and the subjects were monitored for 60 minutes after the biopsies to ensure that they are stable without signs of any bleeding or other complications. Using standard large size biopsy forceps, two sigmoid colon mucosal biopsies were collected from 15 IBS-C, 21 IBS-D patients and 25 healthy controls. Ten of these subjects (5 controls and 5 IBS patients) were randomly selected to have a second, IRB-approved sigmoidoscopy 2-3 months after the first collection in order to assess data reproducibility.

### Array Processing and Pre-Processing of the Data

Upon collection, colon biopsy samples were immediately submerged in 5 volumes of RNAlater solution (Ambion, Austin, Tex.) and stored at −20° C. until further analysed. Tissue was homogenized in a mixer mill 501 (Qiagen, Venlo, The Netherlands) in RLT cell lysis buffer (Qiagen), followed by RNA extraction from the disrupted cells using the RNeasy mini kit (Qiagen) with DNase treatment on the column. One μg of total RNA was biotin labelled and hybridized on Human Genome U133 Plus 2.0 GeneChip microarrays according to the instructions of the provider (Affymetrix, Santa Clara, Calif.). Given the large number of samples (n=132), this processing in the laboratory was performed in 4 different batches, each comprising samples from both IBS and healthy subjects. Gene expression summary values for raw Affymetrix GeneChip data were computed using the gcRMA algorithm (Wu et al. 2004), which does background adjustment, quantile normalization and summarization, taken GC affinities into account. PANP (Warren et al., 2006) was used for calling the detection of genes absent or present, and filtered genes when they were called present in at least 50% of the samples in one treatment group (McClintick and Edenberg, 2006). An effect of the different sample processing batches remained apparent after normalization. This technical source of variation was corrected for by modelling the expression levels in function of batch of origin in a one-way ANOVA, and using the residuals of this model for all subsequent analyses. Finally, to avoid misleading results due to pseudoreplication (Hurlbert 1984), the expression values of the replicated samples were averaged per patient for the SAM and PAM analyses (see below).

### Assessing Concordance of Repeated Measurements

To quantify the sample reproducibility over time and tissue space for the same patient, concordance coefficients (Lin 1989) were calculated for the 1000 most variable gene probes, as well as for the set of 32 gene probes that were found to be predictive for IBS disease status in the PAM analysis.

### Testing for Differentially Expressed Genes

Significance analysis of microarrays (SAM) was applied to identify differentially expressed genes in IBS-diseased versus healthy persons, using a D of 0.05 (Tusher et al., 2001). An alternative, more rigorous statistical model was also applied on the raw data, (i.e. preprocessed data of all biopsy samples before batch correction), by application of mixed ANOVA with batch and disease status as fixed and patient as a random effect, and with FDR correction (Storey et al., 2003).

### Classification

For disease status prediction, Predictive Analysis of Microarrays (PAM) was applied, which is an enhanced variant of nearest centroid classification using shrunken centroids (Tibshirani et al., 2002). Samples from 8 IBS patients and 8 healthy subjects were kept independent from the model building step to assess the model's predictive power so as to check for possible overfitting.

### Hierarchical Clustering

To identify an underlying structure in the molecular signatures, hierarchical clustering (Spotfire DecisionSite 8.2 software) was applied on a set of 16 gene probes that were selected both in the PAM and SAM analysis.

### URLs

SAM software is available at http://www-stat.stanford.edu/˜tibs/SAM/.

PANP software is available at http://people.brandeis.edu/˜dtaylor/PANP/.

### Results & Discussion

### Sample Analysis

Biotin-labelled total RNA prepared from each of the colon biopsy samples was hybridised on Human Genome U133 Plus 2.0 GeneChip microarrays (Affymetrix). Pre-processing of the generated raw data files, including background adjustment, data normalization and transformation, and correction for technical batch variation, revealed profiles of gene expression summary values for each sample that were used in all further analyses.

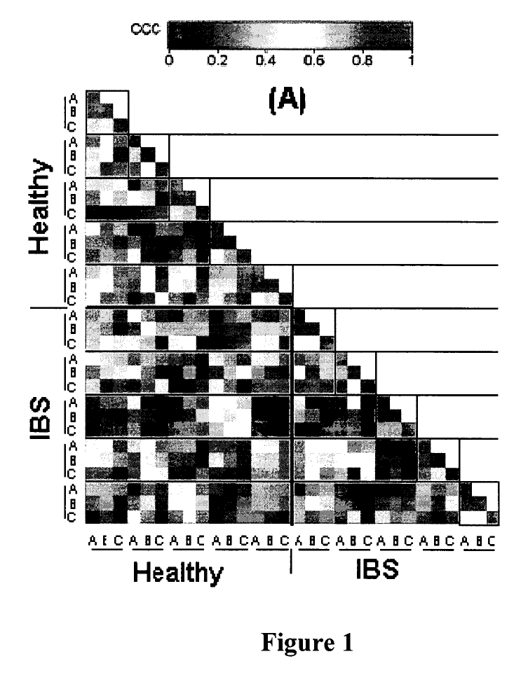

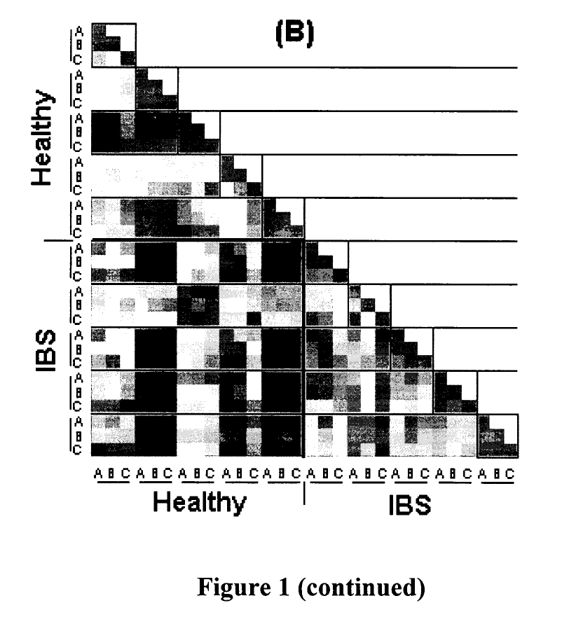

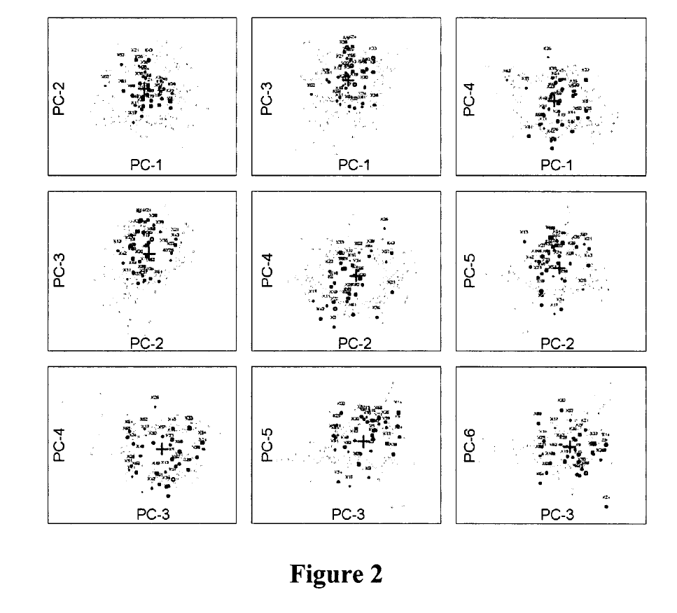

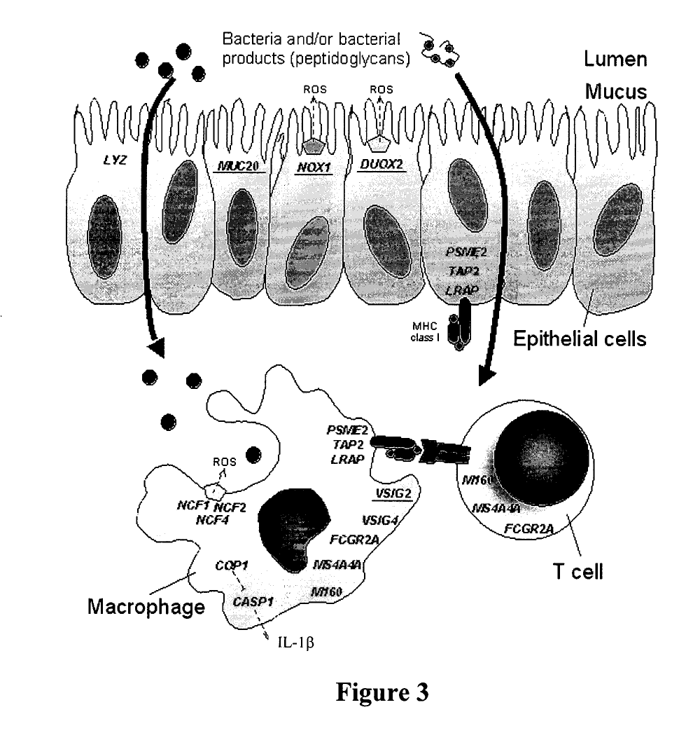

A prerequisite for any useful biomarker is the reproducibility of the gene expression profiles within individual subjects. The concordance coefficient, which measures how well a set of points matches the identity line (Lin 1989), was measured between repeated samples of the same patient using the 1,000 most variable gene probes on the microarray. Both the concordance between two simultaneously collected samples (0.7±0.03), as well as between two samples collected from the same person with an interval period of 3 months (0.41±0.03) significantly exceeded the overall concordance (0.25±0.12; Mann-Whitney U test; respectively W=3510, p<0.0001 and W=6744, p<0.0001). No significant differences in the concordance values were observed between IBS patients and healthy controls (FIG. 1A). Since the overall expression profiles of sigmoid colon biopsies were relatively stable for two site and two time sample collections, the gene probe expression levels of the two collected colon samples per patient was averaged for the subsequent analyses like significance analysis of microarray (SAM) and classification (see below). Moreover, the selection of the subgroup for repeat sample collection was representative of the original study group. This was assessed by randomly selecting 10 individuals for the repeat sample collection: 5 healthy controls and 5 IBS patients, without considering of any selection criteria. A posteriori, it was verified whether this subgroup of subjects was representative for the whole cohort using spectral map analysis. The graphical output of this analysis—summarizing the combined effect of the six first principal components—shows that the subjects selected for repeat sampling are indeed representative for the whole cohort (FIG. 2).

Next, a search was performed for differentially-expressed genes between IBS patients and healthy controls, using the SAM algorithm (Tusher et al., 2001). At a 5% false discovery rate, 25 gene probe sets were found to be differentially expressed between IBS and healthy persons at a significance level of 0.1 for the q-values. These 25 significant (q<0.1) gene probe sets represented 20 different genes: 4 up-regulated and 16-down regulated in IBS patients compared to healthy controls (Table 1). An alternative statistical approach was also applied on the normalized raw data. This was a mixed ANOVA model with batch and disease status as fixed and patient as random effect, and with false discovery rate correction (Storey et al., 2003). This analysis resulted in a very similar list of genes with q-values comparable to the SAM analysis (Table 1). The genes that were significantly differentially expressed reflected subtle changes in expression levels: only a few of the significant genes had changes in expression level >1.5-fold between IBS patients and healthy controls.

TABLE 1

| | | | | | |
|---|---|---|---|---|---|
| | Gene | q-value | q-value | | |
| Probe set | symbol | SAM | ANOVA | Fold change | Gene annotation |

HIGHER expression in IBS patients
versus controls

| | | | | | |
|---|---|---|---|---|---|
| 225809_at | IBS1 | 0.018 | 0.03 | 1.41 | DKFZP564O0823 (IBS1) |
| 204687_at | IBS1 | 0.018 | 0.01 | 1.24 | DKFZP564O0823 (IBS1) |
| 226622_at | MUC20 | 0.018 | 0.05 | 1.52 | Mucin 20 |
| 229369_at | VSIG2 | 0.023 | 0.01 | 1.20 | V-set and immunoglobulin |
| | | | | | domain containing, 2 |
| 231941_s_at | MUC20 | 0.030 | 0.07 | 1.47 | Mucin 20 |
| 200884_at | CKB | 0.039 | 0.05 | 1.29 | Creatine kinase, brain |

LOWER expression in IBS
patients versus controls

| | | | | | |
|---|---|---|---|---|---|
| 223655_at | M160 | 0.018 | 0.05 | 0.69 | Scavenger receptor |
| | | | | | cysteine-rich type 1 protein |
| | | | | | M160 (CD163 antigen-like |
| | | | | | 1) |
| 204787_at | VSIG4 | 0.023 | 0.05 | 0.66 | V-set and immunoglobulin |
| | | | | | domain containing, 4 |
| 211368_s_at | CASP1 | 0.030 | 0.05 | 0.75 | Caspase 1, apoptosis- |
| | | | | | related cysteine peptidase |
| | | | | | (interleukin 1, beta, |
| | | | | | convertase) |
| 205147_x_at | NCF4 | 0.030 | 0.05 | 0.70 | Neutrophil cytosolic factor |
| | | | | | 4, 40 kDa |
| 1555745_a_at | LYZ | 0.030 | 0.11 | 0.48 | Lysozyme |
| 205968_at | KCNS3 | 0.039 | 0.10 | 0.66 | Potassium voltage-gated |
| | | | | | channel, delayed-rectifier, |
| | | | | | subfamily 5, member 3 |
| 211367_s_at | CASP1 | 0.039 | 0.06 | 0.75 | Caspase 1, apoptosis- |
| | | | | | related cysteine peptidase |
| | | | | | (interleukin 1, beta, |
| | | | | | convertase) |
| 201762_s_at | PSME2 | 0.045 | 0.01 | 0.84 | Proteasome activator |
| | | | | | subunit 2 (PA28 beta) |
| 211366_x_at | CASP1 | 0.049 | 0.06 | 0.76 | Caspase 1, apoptosis- |
| | | | | | related cysteine peptidase |
| | | | | | (interleukin 1, beta, |
| | | | | | convertase) |
| 219607_s_at | MS4A4A | 0.056 | 0.13 | 0.74 | Membrane-spanning 4- |
| | | | | | domains, subfamily A, |
| | | | | | member 4 |
| 220085_at | HELLS | 0.058 | 0.07 | 0.82 | Helicase, lymphoid-specific |
| 1552703_s_at | COP1 | 0.080 | 0.06 | 0.81 | Caspase 1 dominant- |
| | | | | | negative inhibitor pseudo- |
| | | | | | ICE |
| 203561_at | FCGR2A | 0.080 | 0.08 | 0.77 | Fc fragment of IgG, low |
| | | | | | affinity IIa, receptor |
| | | | | | (CD32) |
| 204023_at | RFC4 | 0.084 | 0.06 | 0.83 | Replication factor C |
| | | | | | (activator 1) 4, 37 kDa |
| 206011_at | CASP1 | 0.084 | 0.11 | 0.77 | Caspase 1, apoptosis- |
| | | | | | related cysteine peptidase |
| | | | | | (interleukin 1, beta, |
| | | | | | convertase) |
| 216237_s_at | MCM5 | 0.084 | 0.08 | 0.76 | MCM5 minichromosome |
| | | | | | maintenance deficient 5, |
| | | | | | cell division cycle 46 (S. cerevisiae) |
| 225973_at | TAP2 | 0.084 | 0.16 | 0.71 | Transporter 2, ATP-binding |
| | | | | | cassette, sub-family B |
| | | | | | (MDR/TAP) |
| 219759_at | LRAP | 0.084 | 0.21 | 0.39 | Leukocyte-derived arginine |
| | | | | | aminopeptidase |
| 218585_s_at | DTL | 0.084 | 0.11 | 0.79 | Denticleless homolog |
| | | | | | (Drosophila) |

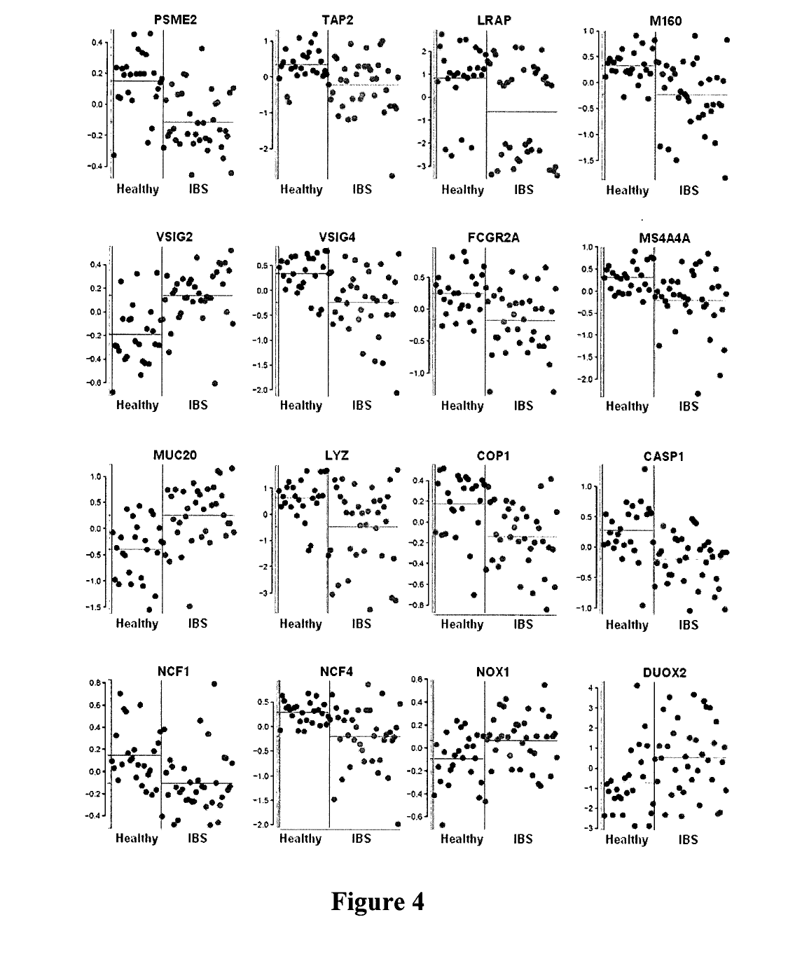

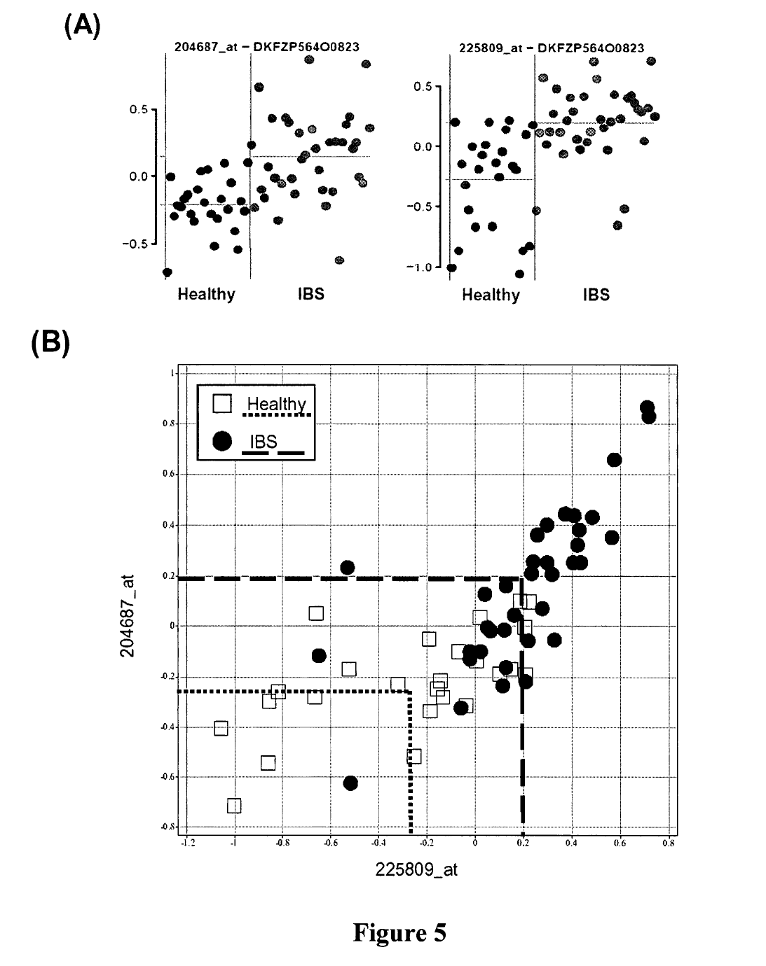

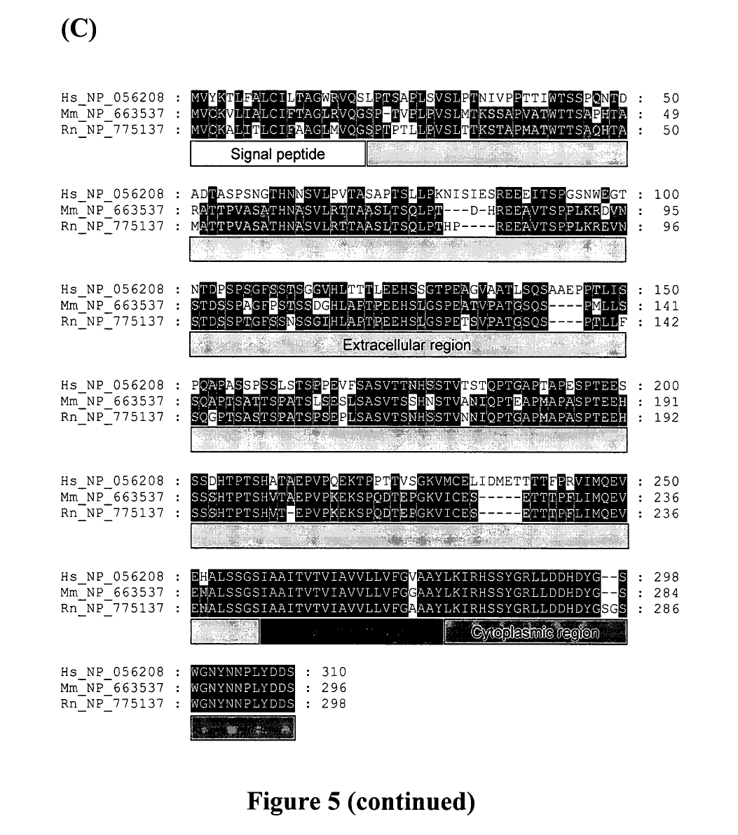

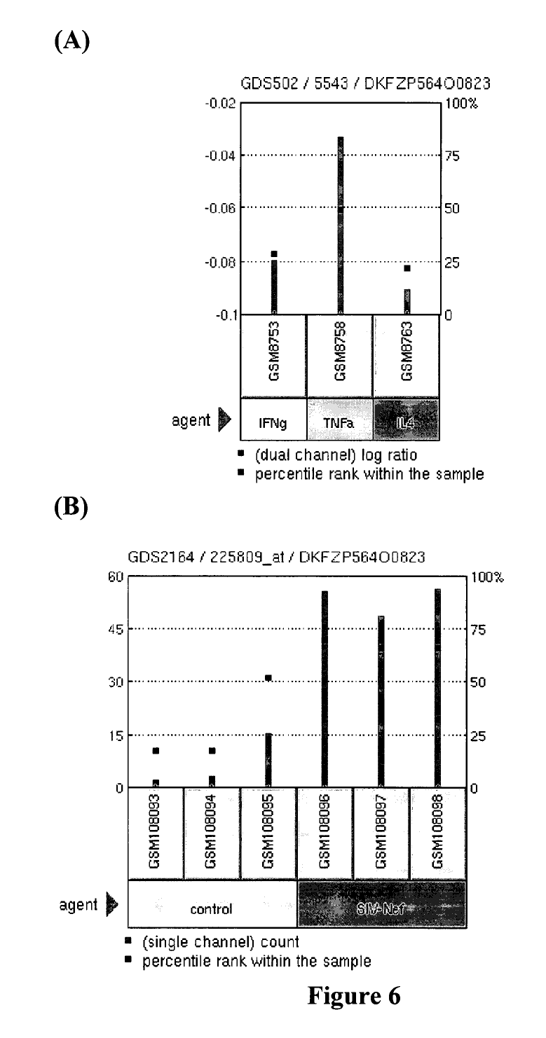

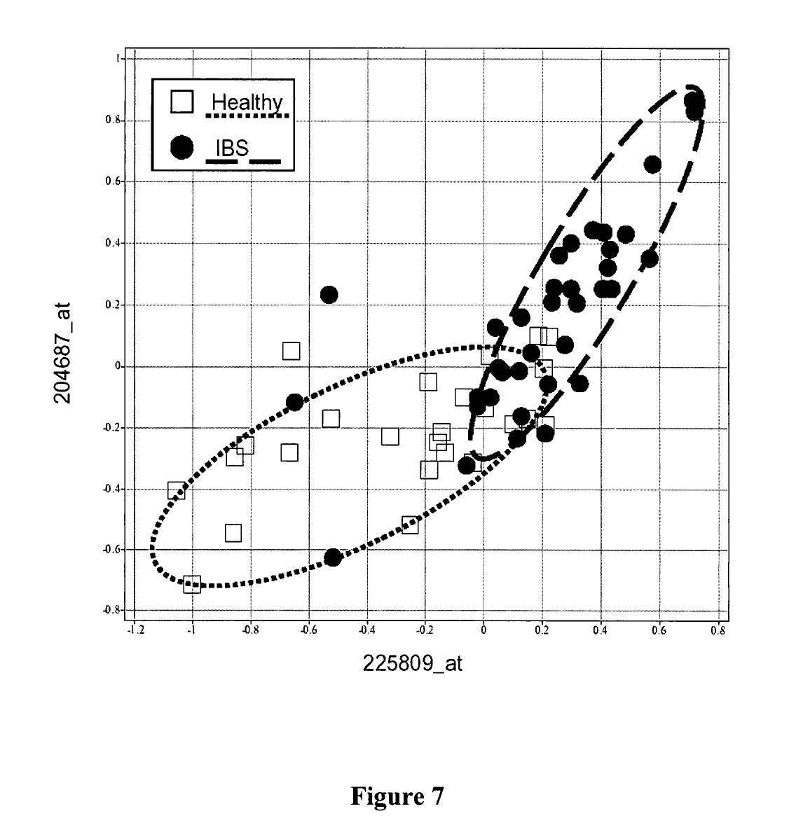

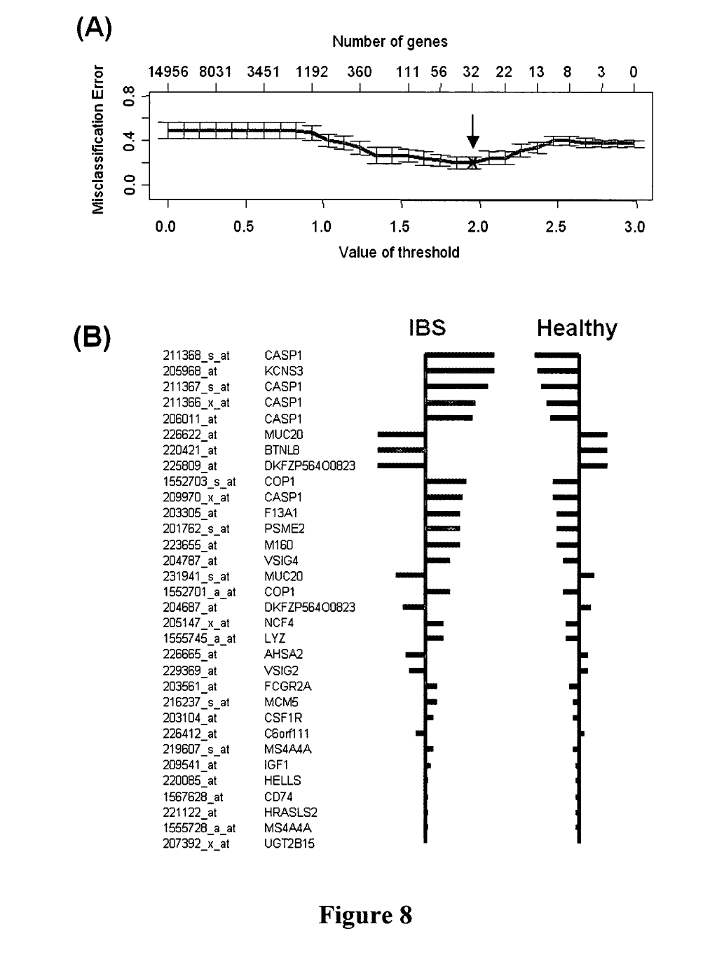

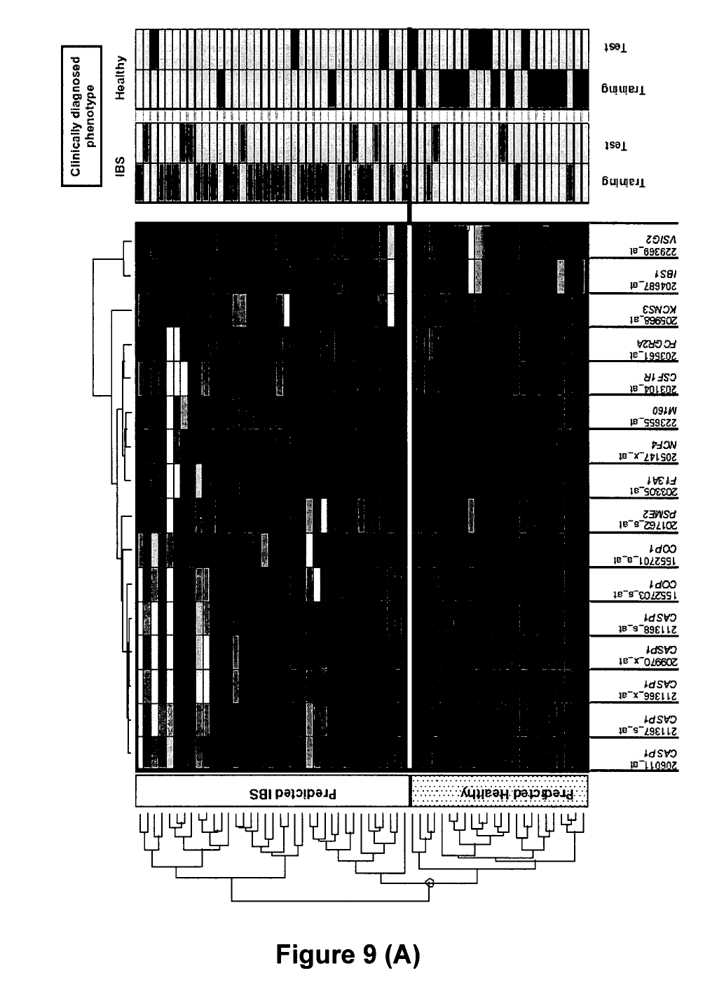

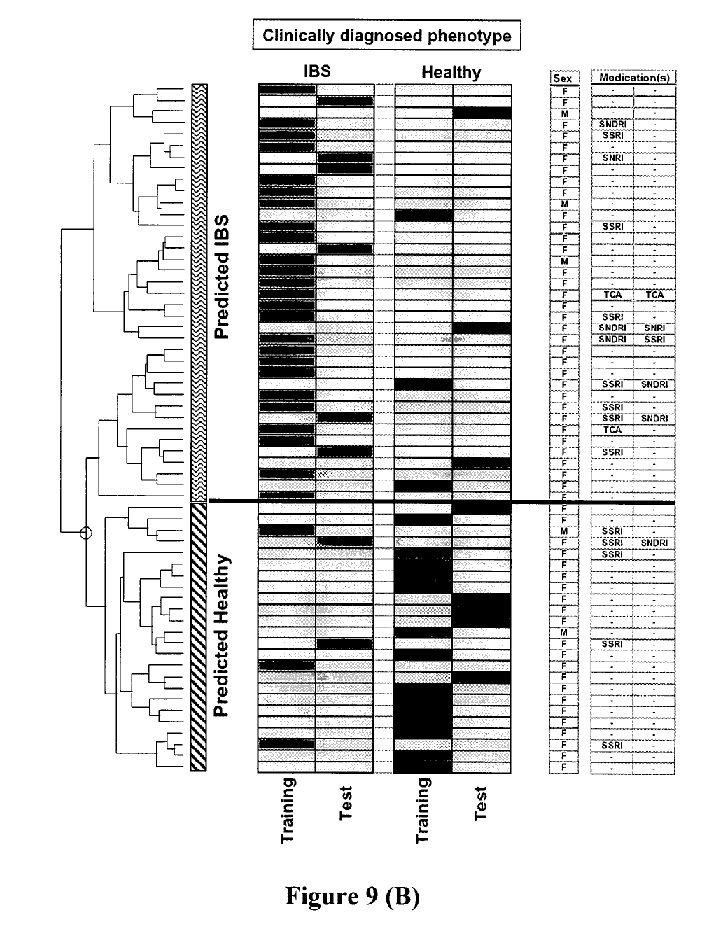

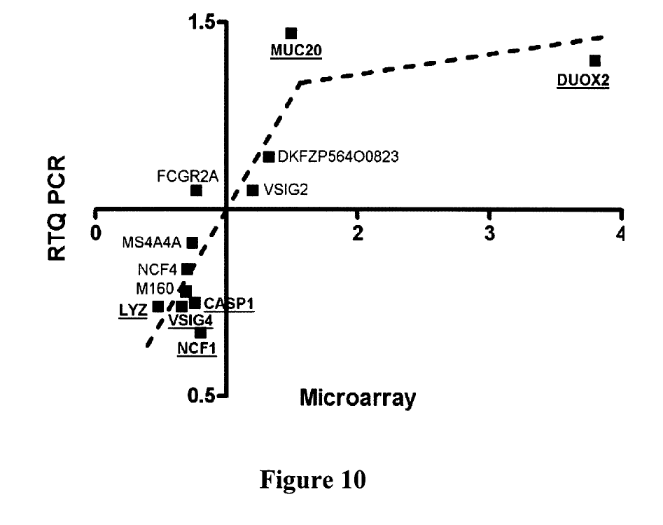

It is remarkable to note that the majority of the 20 identified significant genes play a role in the immune response or the host defense system against microbial invasion. A schematic overview of the differentially expressed genes in mucosal colon of IBS patients versus healthy controls is provided in FIG. 3. Plots of the expression levels of some of the significant genes with differential expression between IBS diseased and healthy persons are shown in FIG. 4.

### Alterations in Genes Affecting Antigen Processing

At least three genes with significantly lower expression levels in the colonic mucosa of IBS patients play an essential role in the pathway of antigen processing and presentation by the major histocompatibility I complex: PSME2 (proteasome activator subunit 2, PA28 beta), TAP2 (transporter 2, ATP-binding cassette, subfamily B), and LRAP (leukocyte-derived arginine aminopeptidase). PA28 is essential in the assembly of the cytosolic immunoproteasome complex, that is responsible for antigen processing of class I major histocompatibility complex (MHC) peptides (Preckel et al., 1999). TAP2 forms, together with TAP1, a heterodimeric transmembrane ATP-binding-cassette (ABC) transporter in the endoplasmic reticulum (ER) membrane that is essential for the delivery of antigenic peptides from the cytosol into the ER, where these peptides are loaded onto MHC class I molecules. TAP2, unlike TAP1, is very unstable in isolation, and the biogenesis of functional TAP depends on the assembly of pre-existing TAP1 with newly synthesized TAP2 but not vice versa, suggesting that mainly TAP2 expression regulates the number of active transporter molecules (Keusekotten et al., 2006). In the ER, MHC class I molecules rely on aminopeptidases to trim precursors to antigenic peptides. LRAP, also named ER aminopeptidase 2 (ERAP2), is one of the key enzymes responsible for the hydrolysis of N-terminal amino acids of proteins or peptide substrates (Saveanu et al., 2005). Together, the significantly lower expression levels of PSME2, TAP2, and LRAP in mucosa of the colon of IBS patients strongly suggest that the functional activity in MHC class I antigen presentation is modulated in these patients.

### Alterations in Genes Controlling Immune Response

In addition, 6 other significantly altered genes in our study are implicated in the immune response: VSIG2, VSIG4, FCGR2A, MS4A4A, M160, and MUC20 (see Table 1 for respective q-values). The expression of VSIG4, a member of the family of V-set and immunoglobulin domain containing proteins (VSIG), is decreased, while the expression of another closely related family member, VSIG2, is higher in the mucosa of the colon of these subjects. The significance of the simultaneous but opposite alteration in gene expression of VSIG4 and VSIG2 in IBS patients is highly interesting given the recent discoveries on the function of these genes. The functional role of all VSIG family members has not yet been well studied, but VSIG4 appears to be critical in the regulation of an immune response mediated by phagocytosis and/or antigen presentation (Kim et al., 2005). Another significant gene alteration provides additional evidence for a modulated immune response system in the colon of IBS patients is FCGR2A (CD32), which encodes the immunoglobulin Fc receptor. These receptors are essential in the protection of the organism against foreign antigens by removing antigen-antibody complexes from the circulation. Fc receptors are present on monocytes, macrophages, neutrophils, natural killer (NK) cells, and T and B lymphocytes, and they participate in phagocytosis of immune complexes and modulation of antibody production by B cells (Unkeless J C, 1989). The expression of MS4A4A and CD163 molecule-like 1 (M160) are also lower in IBS patients. MS4A4A is a 13 subunit homolog of another immunoglobulin receptor, and CD163 molecule-like 1 (M160) is a membrane-anchored member of the scavenger receptor cysteine-rich superfamily that is mainly expressed in cells associated with the immune system.

### Alterations in Genes Involved in Local Defense Mechanisms

The expression of the cell surface associated mucin 20 protein (encoded by MUC20), on the other hand, is elevated in IBS patients. This gene is known to be predominantly expressed in the kidney, and an elevated expression has been described in epithelial cells from the proximal renal tubules in IgA nephropathy patients as well as several renal injury models (Higuchi et al., 2004). Moreover, stimulation of a renal tubular epithelial cell line with proinflammatory substances such as lipopolysaccharide (LPS), phorbol 12-myristate 13-acetate (PMA), or tumor necrosis factor alpha significantly increases MUC20 mRNA expression. The elevated MUC20 expression found in the colonic mucosa of IBS patients might reflect a response to a local injury or inflammation.

### Alterations in Genes Involved in Host Defence Response to Pathogens

At least 4 of the differentially expressed genes (LYZ, CASP1, COP1, NCF4) (Table 1) are involved in the host defense response to pathogens in the colon. The expression level of the anti-microbial agent lysozyme (LYZ), whose natural substrate is the bacterial cell wall peptidoglycan, is significantly lower in IBS patients, suggesting that there may be the biological basis for a compromised innate immunity in the colon of IBS patients; this function requires further study. Paneth cells are secretory epithelial cells of the small intestinal mucosa, and a major source of anti-microbial peptides including lysozyme. These antimicrobial defenses may also be recruited to sites of inflammation in the colon through metaplasia of Paneth cells, most likely as a mucosal innate immunity response mechanism (Wehkamp et al., 2006). Whether the mucosal innate immune system in the colon is affected in IBS patients will require further study. Pro-inflammatory cytokines, such as interleukin-1β (IL-1β and gamma interferon (IFN-γ), are important components of the antimicrobial defense system. IL-18 is an IFN-γ-stimulating factor, and plays an important role in defense against a variety of gram-positive and -negative bacterial pathogens. The synthesis of IL-1β and IL-18 depends upon the proteolytic cleavage of their precursor proteins (pro-IL-1β and pro-IL-18) by the cysteine protease caspase 1 (CASP1), also known as interleukin-1β (IL-1β) converting enzyme. Caspase-1 deficient mice indeed have a major defect in IL-18 and IL-1β production in vivo, and this is accompanied by a resistance to lethal doses of endotoxin/LPS (Li et al., 1995). These mice are also two- to threefold more susceptible to lethal Escherichia coli infection than wild-type mice due to a failure of the innate host defense mechanism (Joshi et al., 2002). Caspase-1 dominant negative inhibitor (COP1), also known as pseudo-ICE, interacts physically with caspase-1 to block its activation, and hence, the secretion of IL-1β and IL-18.

The expression of both CASP1 and COP1 is decreased in the mucosal colon of IBS patients. The genes for CASP1 and COP1 are contiguous on chromosome 11q, and it has been suggested that both genes are under similar transcriptional regulation, based on their identical tissue distribution (Druilhe et al., 2001). The effects of the altered mRNA expression of CASP1 and COP1 on the protein expression level and subsequently on the production of IL-1β and IL-18 are, however, unknown. In the samples of our study population, IL-1β was not differentially expressed, but an increased rectal mucosal mRNA expression of IL-1β has been reported recently in acquired post-infectious IBS patients (Gwee et al., 2003).

The NADPH oxidase complex was originally identified and characterized in phagocytes, where it plays an essential role in non-specific host defense against microbial organisms, by catalysing the generation of an oxidative burst of superoxide from oxygen and NADPH. The structure of NADPH oxidase consists of 2 membrane-bound elements (gp91^{phox} and p22^{phox}, encoded by CYBB and CYBA, respectively), three cytosolic components (p67^{phox}, p47^{phox} and p40^{phox}, encoded by NCF2, NCF1, and NCF4), and a low molecular weight G protein (either rac 1 or rac2, encoded by RAC1 and RAC2). Activation of the enzyme complex is associated with the migration of the cytosolic components to the cell membrane, so that the complete oxidase can be assembled (DeCoursey and Ligeti, 2005). The lower expression of NCF1 and NCF4 in the mucosal colon of IBS patients may thus cause a shortage of cytosolic components transported to the membrane-bound elements of the NADPH oxidase enzyme complex, leading to a diminished activity of phagocyte-expressed NADPH oxidase. The essential catalytic core of the oxidase, gp91^{phox} (nowadays also called Nox2), belongs to a family of several very similar oxidases. Homologues of the NADPH oxidase complex were identified in numerous non-phagocytic cell types, including the Nox1 enzyme complex that is predominantly expressed in surface mucous epithelial cells of the colon (Kikuchi et al., 2000). The different enzyme complex homologues can be distinguished at the molecular level based on their subunits composition. Compared to the Nox2 complex that is expressed in phagocytes, the Nox1 complex shares the p22^{phox}(CYBA) subunit, but the (CYBB), p47^{phox} (NCF1), and p40^{phox} (NCF4) subunits are exchanged for NOX1, NOXO1, and NOXA1, respectively. Interestingly, a borderline elevated NOX1 expression in the colonic mucosa of at least a subset of IBS patients was found for several NOX1 probe sets on the microarray (206418_at; 207217_s_at; 210808_s_at). It has been demonstrated that human colonic epithelial cells induce Nox1 expression and up-regulate superoxide production in response to IFN-γ (Kuwano et al., 2006) and to flagellin from Salmonella enteritidis (Kawahara et al., 2004). Helicobacter pylori lipopolysaccharide, known to cause a persistent inflammation and enhanced Tₕ1 immune response in human gastric mucosa, also stimulates the mRNA expression of NOX1 and NOXO1 in guinea pig gastric mucosal cells, followed by an upregulation of superoxide generation (Kusumoto et al., 2005). Another member of the family of NOX/DUOX oxidase genes is dual oxidase 2 (DUOX2), which is expressed all along the digestive tract, with the highest levels found in the epithelial cells of mucosal surfaces of caecum and sigmoid colon (El Hassani et al., 2005). DUOX2 (but not DUOX1) mRNA expression levels were also found to be highly increased in the sigmoid colon mucosal biopsies of IBS patients (219727_at, q=0.15, 3.8-fold increase, which is the largest fold-change of all genes). This finding might be somewhat surprising, as DUOX2 is thought of as an inducible rather than a constitutively expressed dual oxidase (in contrast to DUOX1), and because DUOX2 generates a robust, self-limited response during infection or inflammation (Harper et al., 2005). Dual oxidases contain both an NADPH oxidase domain, responsible for H₂O₂ production, as well as a heme peroxidase domain that is closely related to several peroxidases including myeloperoxidase and lactoperoxidase. These oxidases provide an epithelial source of reactive oxygen species (ROS) such as superoxide and H₂O₂, which have an essential role in host defense mechanisms (Geiszt et al., 2003; El Hassani et al., 2005). Pro-inflammatory stimuli such as IFN-γ induce the expression of DUOX2, and lead to elevated H₂O₂ production (Harper et al., 2005). A direct role for dual oxidase in gut immunity was demonstrated in Drosophila: adult flies in which dual oxidase expression was silenced showed a marked increase in mortality even after a minor infection through ingestion of microbe-contaminated food. This effect could be reversed by the reintroduction of dual oxidase, demonstrating that the enzyme generates a unique epithelial oxidative burst that limits microbial proliferation in the gut (Ha et al., 2005). It is considered that the elevated DUOX2 expression is indicative for the existence of a mild but chronic inflammatory condition in the colon of IBS patients. Together, the altered expression of several members of multi-subunit NADPH oxidase/dual oxidase enzyme complexes in the colon of IBS patients provide further support for the hypothesis that the host defense response to bacteria and other invaders in the colon is disturbed in IBS patients.

### Upregulation of a Novel Gene in Irritable Bowel Syndrome

Finally, two of the most significantly up regulated probe sets in colon mucosal biopsies of IBS patients (FIG. 5A) represent the same gene that is annotated in the public sequence databases as DKFZP56400823 (NCBI GeneID: 25849). This gene isrenamed IBS1 (Irritable Bowel Syndrome 1) herein. In silico analysis demonstrates that the 5 kb cDNA sequence of IBS1 contains an open reading frame of 930 by that encodes a predicted plasma membrane protein of 310 amino acids (AA), including a signal peptide (20 AA), an extracellular region (238 AA), a transmembrane region (21 AA), and an intracellular region (31 AA).

A mouse homologue for the IBS1 gene is known as RIKEN cDNA 9130213B05, and has been further annotated as a cell surface glycoprotein precursor. In rat, the IBS1 homologue was identified as an up regulated gene in ventral prostate upon castration, associated with apoptosis, and was therefore annotated as Cipar-1 (castration induced prostatic apoptosis-related protein 1) or PARM-1 (prostatic androgen-repressed message 1) (Bruyninx et al., Endocrinology, 1999, 140:4789-4799; Cornet et al., Prostate, 2003, 56:220-230).

Although no literature on the human DKFZP56400823 gene is yet available in PubMed, the Gene Expression Omnibus (http://www.ncbi.nlm.nih.gov/geo/) comprises experimental microarray data with significantly altered expression of the human DKFZP56400823 gene that point towards a role in inflammation and immune response. In a first study, primary human endothelial cells derived from different tissues were challenged with inflammatory and immune cytokines. The expression of DKFZP56400823 was increased in primary colon endothelial cells upon exposure to TNFα as compared to exposure to IFNγ or IL-4 (FIG. 6A). The latter two are primarily associated with T helper cell subsets, whereas TNFα is a pleiotropic cytokine with a critical function in both inflammatory and immunological responses. As these data on the colon endothelial cells form only a part of a large study, the publication on these experiments does not include the results on DKFZP56400823, but rather focuses on well-known genes (Sana et al, Cytokine 2005; 29:256-269).

In a second study, gene expression was assessed in Jurkat CD4+ T cells following induction of the Nef protein from the simian immune deficiency virus (SIV) (Ndolo et al, Virology 2006; 353:374-387). The Nef protein is expressed early in SIV (and HIV) infections, and down-regulates major histocompatibility complex class I (MHC-I) molecules from the cell surface—thereby facilitating immune evasion. The microarray experiment reveals that—among many other genes that are well characterized—the expression of the DKFZP56400823 gene is upregulated by SIV-Nef (FIG. 6B).

These two latter studies in the literature are conceptually in line with our results that also point towards alterations in immune response in the colon of IBS patients compared to controls. The hypothesized biological link of the DKFZP56400823 gene to IBS via an effect on immune response requires further functional characterization.

The gene is mainly expressed in colon and placenta, but is also found in many other tissues. Amino acid sequence alignment of the human, mouse, and rat homologues demonstrate a highly conserved sequence in the transmembrane and intracellular regions (95% and 94% amino acid sequence identity among the three species in these regions, respectively), but less homology in the extracellular region (51% AA sequence identity) (FIG. 5C).

As no information on the functional role of IBS1 is currently known, the consequence of the increased expression level in the colon of IBS patients is unclear. However, it is striking to note that two independent gene probe sets on the microarray identified this gene as the most significant in two independent analyses (SAM and mixed ANOVA) and that a relatively good discrimination of IBS from and health is possible solely based on these two probe sets (FIG. 7, FIG. 5B).

It is unclear whether the differentially expressed genes are the cause or rather the consequence of IBS. Because IBS is likely a complex multifactorial disorder, many genes may be involved, each with a relatively small contribution to the overall phenotype. Our results showing mainly subtle changes in expression of many genes in the colonic mucosa of IBS patients, support this concept. The altered expression of a specific gene was, in some cases, observed in only a subset of the IBS patients; this may reflect heterogeneity of the underlying molecular mechanisms with a common phenotype. Most of the genes with significant changes in expression are implicated in similar functional cellular processes involved in the host response to intraluminal antigen or bacterial invasion or their pro-inflammatory effects. Given this degree of mechanistic specificity in the identified genes, it is considered unlikely that they represent false positive associations picked up by chance using the two applied statistical methodologies.

### Potential for Molecular Diagnosis of IBS Based on Mucosal Gene Expression Profiling

Because of its clinical relevance, the predictive power of a molecular diagnosis of IBS based on sigmoid colon expression profiles was subsequently assessed. The 61 subjects were therefore randomly divided into a training set and a test set. The training set (n=45) comprised 17 healthy subjects and 28 IBS patients (16 IBS-D, 12 IBS-C); the test set (n=16) comprised the remaining 8 healthy controls and 8 IBS patients (5 IBS-D, 3 IBS-C). Subsequently, two different and independent classification analysis methods were applied that that have been shown to perform well on datasets with many measurements and relatively few samples, as they are quite robust against overfitting. First, using Prediction Analysis for Microarrays (PAM) (Tibshirani et al., 2002) on the validation set, a 32 gene probe sets signature was obtained (FIG. 8) with an average cross-validation misclassification rate of 22% (respectively 13/17 and 22/28 correctly classified for healthy and IBS subjects). Using this molecular signature on the independent samples in the test set, PAM correctly predicted the disease status of 75% of the participants, with an equally accurate prediction for both diseased and healthy persons. Overfitting did not seem to be an issue as the misclassification rates were similar for the training set (22%) and the test set (25%).

In order to further validate the molecular signature of 32 gene probe sets, reproducibility was determined. Thus, the molecular signature was highly reproducible between repeated samples in the same patient for the simultaneously collected samples (concordance 0.76±0.05), as well as between two samples collected with an interval period of 2-3 months (concordance 0.67±0.04). Within patient concordance significantly exceeded the overall concordance between participants (concordance −0.02±0.02; Mann-Whitney U test with unequal variances; respectively W=3938, p<0.0001 and W=7683, p<0.0001) (FIG. 1B).

Finally, it was attempted to further reduce the number of probes in the molecular signature through the selection of gene probes that were identified in common by the PAM and a SAM analysis performed on the samples of the training set only. The resulting set of 16 probes, representing 11 different genes, was then used in an unsupervised classification method, hierarchical clustering, including all 61 subjects of the study (i.e. both the training and test set). This clustering method groups on the one hand the gene probes with a similar gene expression profile amongst the different subjects together, and on the other hand also groups the colonic samples with a similar expression profile over the 16 gene probes together into a cluster hierarchy, using average linkage and correlation as similarity measure (FIG. 9A). At the first level of hierarchy of the gene probes (X-axis), a clear distinction was made between the gene probe sets that are up- versus down-regulated in IBS patients versus healthy controls. At the first level of hierarchy of the subjects (Y-axis), a subset of IBS patients is separated from all other subjects, which appears to be strongly determined by their low expression levels of F13A1, NCF4, M160, CSF1R, and/or FCGR2A. The second hierarchical clustering level further separates two groups, largely corresponding to the group of IBS patients and the healthy individuals. As might be expected for the samples of the training set that were used to select the gene probe sets for classification, a good separation was observed (respectively 14/17 and 25/28 correctly classified for the healthy subjects and the IBS patients). Overall, for the test set subjects, 11 out of 16 were correctly classified (69%), which is in line with the results of the above described PAM analysis. The positive predictive value (i.e., the probability that people with the molecular signature are indeed IBS patients: 6/8) was 75%, while the negative predictive value (i.e., the probability that people lacking the molecular signature are not IBS patients: 5/8) was 63%. Future studies will allow to more precisely determine the predictive value, the sensitivity and specificity, but it is already clear that these molecular signatures in the mucosal colon have the power to identify the majority of IBS patients. This finding demonstrates the possibility to define specific subgroups of IBS patients based on a common molecular signature, and hence paves the way towards more personalized medicine. The assessment of molecular signatures in mucosal colon biopsies provides a new complementary tool to help a clinician deciding on the diagnosis of IBS, and upon the most beneficial therapeutic strategy for an individual patient.

A potential influence of gender or drug treatment on the results should be considered. A graphical presentation of the distribution of gender and drug treatment over the cohort is included in FIG. 9B, suggesting that any potential effect on the classification analysis by these potential confounders would have been only marginal. This was confirmed by an additional mixed ANOVA analysis using the set of signature genes. It was found that:
a. medication did not affect the expression level of these genes (all q>0.7 for testing medication effect)
b. there was no interaction between medication and disease indicating that the gene expression was not influenced by medication depending on disease status, that is health versus IBS (all q>0.1 for testing interaction between medication and disease status)
c. gender did not affect the expression level of these signature genes (all q>0.9 for testing medication effect) or the difference between health and IBS patients (all q>0.9 for testing interaction between gender and disease status).

### Confirmatory Data for the Changes in Gene Expression Found by Microarray Analysis by Independent Technology, Reverse-Transcription Quantitative Polymerase Chain Reaction (RTQ-PCR)

12 genes that were identified from the microarray data analysis were selected for confirmatory analysis by validated fluorogenic TaqMan gene expression assays-on-demand (Applied Biosystems). Normalisation of the TaqMan assay results was done relative to the control SART1 gene, because this gene was found earlier to be stable and is also moderately expressed in colon samples (Camilleri M et al, Gastroenterology 2007). Overall, the data show substantial concordance between Affymetrix microarray and TaqMan data when comparing the fold change in expression level between IBS patients and healthy subjects (FIG. 10). With the exception of the FCGR2A gene, 11 out of the 12 genes analyzed showed the same trend of up- or down-regulated expression in IBS compared to healthy subjects. Significant differences (p<0.05) between IBS and healthy subjects were confirmed in 6 out of the 12 genes, and these represented the genes with the largest fold change values. This level of significance was not achieved for genes with a more subtle fold change difference. This can be explained, in part, by the fact that the TaqMan method only normalizes the gene expression data versus a single reference gene (SART1 in our assay), whereas the microarray method allows normalization of the expression level of each individual gene against all other genes on the microarray. This means that even relatively small gene expression variations of the SART1 gene will introduce noise (and variation) in the normalized expression level of the genes of interest using the RTQ-PCR technology. Thus, although the TaqMan technology has some advantages with regard to sensitivity compared to microarrays (i.e. genes with low expression can be analyzed using RTQ-PCR where microarray technology may fail), it is clear that relatively larger intra-group variations are often found for TaqMan assays as compared to the microarray analyses.

In summary, the data generated by TaqMan assays largely confirm the microarray data with regard to the fold change levels of the individual genes.

In conclusion, several differentially expressed genes are found in the colonic mucosa of IBS patients. Many of these genes are directed towards a change in host defense mechanisms. Proteins involved in host defense mechanisms, or those encoded by the differentially expressed genes, may represent potential targets for the development of novel drugs for IBS. The most significant gene with an elevated expression in IBS patients encodes for a poorly characterized membrane protein (DKFZP564O0823) of unclear function, for which the name IBS1 is proposed. Molecular signatures of gene expression in the colon, based on a limited set of genes, may be predictive of IBS disease status. These gene expression profiles are stable over several months, suggesting that the molecular signatures have potential to improve the diagnosis of IBS and monitor therapeutic effectiveness. At the very least, these biological differences in patients with IBS suggest that there are objective differences in the colon and that the disease does not represent exclusively a disorder of central nervous system perception.

1-22. (canceled)

23. An method for detecting and/or monitoring Inflammatory Bowel Syndrome (IBS) in a subject, said method comprising:
(a) determining, in a biological sample of said subject, the level of gene transcription of an IBS molecular signature gene (IBS-MSG), wherein said IBS-MSG is selected from the group consisting of Irritable Bowel Syndrome 1 (IBS1) represented by SEQ ID NO:15; Caspase 1 dominant-negative inhibitor pseudo-ICE (COP1); Proteasome activator subunit 2 (PA28 beta/PSME2); Coagulation factor XIII, A1 polypeptide (F13A1); Neutrophil cytosolic factor 4, 40 kDa (NCF4); Colony stimulation factor-1 receptor (CSFR1); Scavenger receptor cysteine-rich type 1 protein (M160); potassium voltage-gated channel, delayed-rectifier, subfamily S, member 3 (KCNS3); Lysozome (LYZ); Membrane-spanning 4-domains, subfamily A, member 4 (MS4A4A); Helicase, lymphoid specific (HELLS); Replication Factor C 4(RFC4); minichromosome maintenance deficient 5, cell division cycle 46 (MCM5); Transporter 2, ATP-binding cassette, sub-family B (TAP2); Leukocyte-derived arginine aminopeptidase (LRAP); Denticleless homolog (Drosophila) (DTL); V-set and immunoglobulin domain containing 2 (VSIG2); V-set and immunoglobulin domain containing, 4 (VSIG4) and Mucin 20 (MUC20); and
(b) comparing the level of gene transcription with the level of gene transcription in a normal control sample; and
(c) determining the presence of IBS and/or its status based on the result from step (b).

24. The method according to claim 23, wherein step (a) comprises determining at least two of the IBS-MSGs.

25. The method according to claim 23, wherein said IBS-MSG is selected from the group consisting of VSIG2, VSIG4 and MUC20.

26. The method according to claim 23, wherein step (a) comprises determining the level of gene transcription of:
IBS1 represented by SEQ ID NO:15, COP1, PSME2, F13A1, NCF4, CSF1R, M160, KCNS3 and VSIG2; or
IBS1 represented by SEQ ID NO:15, PSME2, F13A1, NCF4, CSFR1 and VSIG2; or
MUC20, VSIG2 and VSIG4.

27. The method according to claim 23 wherein step (a) further comprises determining the level of gene transcription of at least one of CASP1, FCGR2A and CKB.

28. The method according to claim 23, wherein, in step (c):
an increase in the level of gene transcription of a gene selected from the group consisting of IBS1 represented by SEQ ID NO:15, VSIG2 and MUC20 or
a decrease in the level of gene transcription of a gene selected from the group consisting of COP1, PSME2, F13A1, NCF4, CSF1R, M160, KCNS3, LYZ, MS4A4A, HELLS, RFC4, MCM5, TAP2, LRAP, DTL and VSIG4,
is an indication of the presence of IBS in said subject.

29. The method according to claim 23, wherein the level of gene transcription of the IBS-MSG is determined either at the protein level or at the nucleic acid level.

30. The method according to claim 23, wherein the level of gene transcription of the IBS-MSG is determined using array technology, wherein said array technology comprises oligonucleotide arrays or protein arrays.

31. The method according to claim 23, wherein the level of gene transcription of the IBS-MSG is determined using array technology, either at the oligonucleotide level using probes that specifically bind to a nucleic acid transcribed from the IBS-MSG or at the protein level using specific binding agents that bind to the IBS-MSG polypeptide.

32. The method according to claim 23, wherein the biological sample is selected from the group consisting of blood, urine, saliva, fecal sample, fecal cells, tissue biopsy or autopsy material.

33. The method according to claim 32, wherein the expression levels of the genes are determined using an array of the probes selected from the probes listed in Table 1 or Table 2.

34. The method according to claim 23, wherein the level of gene transcription is determined at the protein level.

35. The method according to claim 34, wherein the protein level is determined using an antibody.

36. The method according to claim 35, wherein said antibody is specific for IBS1 represented by SEQ ID NO:15.

37. A method for identifying a candidate compound for the treatment of CVH, IBS, or a combination thereof, said method comprising:
(a) contacting a cell expressing at least one IBS-MSG, wherein the IBS-MSG is selected from the group consisting of IBS1 represented by SEQ ID NO:15, COP1, PSME2, F13A1, NCF4, CSFR1, M160, KCNS3, LYZ, MS4A4A, HELLS, RFC4, MCM5, TAP2, LRAP, DTL, VSIG2, VSIG4 and MUC20, with a test compound;
(b) determining the expression level of said IBS-MSG in said cell; and
(c) comparing with the expression level of said IBS-MSG to the expression level in a control cell in the absence of said compound;
(d) selecting a test compound that changes the expression level of said IBS-MSG in said cell relative to the expression level in the absence of said compound,
thereby identifying a candidate compound for the treatment of CVH, IBS, or a combination thereof.

38. The method according to claim 37, wherein the IBS-MSG is IBS1 represented by SEQ ID NO:15.

39. A method according to claim 37, wherein the expression level is detected at the nucleic acid level or the protein level.

40. The method according to claim 37, wherein the level of gene expression is determined using an array of oligonucleotide probes that bind to the IBS-MSGs.

41. The method according to claim 37, wherein the expression level is determined at the protein level.

42. The method according to claim 41, wherein the protein level is determined using an antibody.

43. A screening method to identify and obtain a candidate compound for the treatment of CVH, IBS, or a combination thereof, said method comprising;
(a) incubating an IBS-MSG product with the compound to be tested, wherein the IBS-MSG is selected from the group consisting of IBS1 represented by SEQ ID NO:15, COP1, PSME2, F13A1, NCF4, CSFR1, M160, KCNS3, LYZ, MS4A4A, HELLS, RFC4, MCM5, TAP2, LRAP, DTL, VSIG2, VSIG4 and MUC20; and (b) determining the capability of said compound to bind with the IBS-MSG product; wherein a compound capable of binding to the IBS-MSG product is a candidate compound for the treatment of CVH, IBS, or a combination thereof.

44. The method according to claim 43 wherein the IBS-MSG product consists of the polypeptide encoded by said gene or a fragment thereof.

45. A diagnostic kit comprising:
(a) at least one probe that specifically binds to an IBS-MSG selected from the group consisting of IBS1 represented by SEQ ID NO:15, COP1, PSME2, F13A1, NCF4, CSFR1, M160, KCNS3, LYZ, MS4A4A, HELLS, RFC4, MCM5, TAP2, LRAP, DTL, VSIG2, VSIG4 and MUC20; or
(b) at least one agent that specifically binds to an IBS-MSG polypeptide or a fragment thereof, the IBS-MSG being selected from the group consisting of IBS1 represented by SEQ ID NO:15, COP1, PSME2, F13A1, NCF4, CSFR1, M160, KCNS3, LYZ, MS4A4A, HELLS, RFC4, MCM5, TAP2, LRAP, DTL, VSIG2, VSIG4 and MUC20.

46. The diagnostic kit of claim 45, which comprises at least two probes each of which specifically binds to an IBS-MSG or at least two agents, each of which specifically binds to an IBS-MSG polypeptide.

47. A diagnostic kit, consisting of probes or agents, capable of specifically detecting the level of gene transcription of:
IBS1 represented by SEQ ID NO:15, COP1, PSME2, F13A1, NCF4, CSF1R, M160, KCNS3 and VSIG2; or
IBS1 represented by SEQ ID NO:15, PSME2, F13A1, NCF4, CSFR1 and VSIG2; or
MUC20, VSIG2 and VSIG4.


SEQUENCE LISTING (19 sequences)

SEQ ID NO: 1 - DNA, 456 bp, Artificial Sequence
  Affymetrix Probe ID No. 206011_at
aatgctctaaaatccaacactgtgtgagcgcccacatgatattcaaaggaaatgtttattgaaacatttcaaattanaggtttttggattagggatgctaaaccagtaagtatacagctgaattccaatatacaaaaatatctgaaatttgaaatacttctggtagcatgcattttggataagggataatcaagccatatacagaaaatactgaagtaatgcctttcttctggtcagtgcagagcacgttgctcttctcccaaagtttttcatttagaccccttttgagattcatctgatattggcctagaagtggccgtaagactacaaaccctcacttttggatgtttctctccttcacctcagcagggtatatttaaacatagcatgtggtctttccttttaaaattgtgtatgttcccattggtggtataactttataacttgcatgtcttt

SEQ ID NO: 2 - DNA, 311 bp, Artificial Sequence
  Affymetrix Probe ID No. 211367_s_at
gagctgaggttgacatcacaggcatgacaatgctgctacaaaatctggggtacagcgtagatgtgaaaaaaaatctcactgcttcggacatgactacagagctggaggcatttgcacaccgcccagagcacaagacctctgacagcacgttcctggtgttcatgtctcatggtattcgggaaggcatttgtgggaagaaacactctgagcaagtcccagatatactacaactcaatgcaatctttaacatgttgaataccaagaactgcccaagtttgaaggacaaaccgaaggtgatcatcatccaggcc

SEQ ID NO: 3 - DNA, 548 bp, Artificial Sequence
  Affymetrix Probe ID No. 211366_x_at
gcacaagacctctgacagcacgttcctggtgttcatgtctcatggtattcgggaaggcatttgtgggaagaaacactctgagcaagtcccagatatactacaactcaatgcaatctttaacatgttgaataccaagaactgcccaagtttgaaggacaaaccgaaggtgatcatcatccaggcctgccgtggtgacagccctggtgtggtgtggtttaaagattcagtaggagtttctggaaacctatctttaccaactacagaagagtttgaggatgatgctattaagaaagcccacatagagaaggattttatcgctttctgctcttccacaccagataatgtttcttggagacatcccacaatgggctctgtttttattggaagactcattgaacatatgcaagaatatgcctgttcctgtgatgtggaggaaattttccgcaaggttcgattttcatttgagcagccagatggtagagcgcagatgcccaccactgaaagagtgactttgacaagatgtttctacctcttcccaggacattaaa

SEQ ID NO: 4 - DNA, 387 bp, Artificial Sequence
  Affymetrix Probe ID No. 209970_x_at
cgaaggtgatcatcatccaggcctgccgtggtgacagccctggtgtggtgtggtttaaagattcagtaggagtttctggaaacctatctttaccaactacagaagagtttgaggatgatgctattaagaaagcccacatagagaaggattttatcgctttctgctcttccacaccagataatgtttcttggagacatcccacaatgggctctgtttttattggaagactcattgaacatatgcaagaatatgcctgttcctgtgatgtggaggaaattttccgcaaggttcgattttcatttgagcagccagatggtagagcgcagatgcccaccactgaaagagtgactttgacaagatgtttctacctcttcccaggacattaaa

SEQ ID NO: 5 - DNA, 210 bp, Artificial Sequence
  Affymetrix Probe ID No. 211368_s_at
aatgtttcttggagacatcccacaatgggctctgtttttattggaagactcattgaacatatgcaagaatatgcctgttcctgtgatgtggaggaaattttccgcaaggttcgattttcatttgagcagccagatggtagagcgcagatgcccaccactgaaagagtgactttgacaagatgtttctacctcttcccaggacattaaaat

SEQ ID NO: 6 - DNA, 206 bp, Artificial Sequence
  Affymetrix Probe ID No. 1552703_s_at
ttccatgggtgaaggtacaataaatggcttactggatgaattattacagacaagggtgctgaaccaggaagagatggagaaagtaaaacgtgaaaatgctacagttatggataagacccgagctttgattgactccgttattccgaaaggggcacaggcatgccaaatttgcatcacatacatttgtgaagaagacagttacctgg

SEQ ID NO: 7 - DNA, 55 bp, Artificial Sequence
  Affymetrix Probe ID No. 1552701_a_at
aggtccgatacctggaaattagcttagtacacaagactcccaattactattttct

SEQ ID NO: 8 - DNA, 295 bp, Artificial Sequence
  Affymetrix Probe ID No. 201762_s_at
caacacctgatccccaagattgaagatggaaatgattttggggtagcaatccaggagaaggtgctggagagggtgaatgccgtcaagaccaaagtggaagctttccagacaaccatttccaagtacttctcagaacgtggggatgctgtggccaaggcctccaaggagactcatgtaatggattaccgggccttggtgcatgagcgagatgaggcagcctatggggagctcagggccatggtgctggacctgagggccttctatgctgagctttatcatatcatcagcagcaacc

SEQ ID NO: 9 - DNA, 454 bp, Artificial Sequence
  Affymetrix Probe ID No. 203305_at
gtccttcacatcaccattttgagacctcagcttggcactcaggtgctgaagggtaatatggactcagccttgcaaatagccagtgctagttctgacccaaccacagaggatgctgacatcatttgtattatgttccaaggctactacagagaaggctgcctgctatgtatttgcaaggctgatttatggtcagaatttccctctgatatgtctagggtgtgatttaggtcagtagactgtgattcttagcaaaaaatgaacagtgataagtatactgggggcaaaatcagaatggaatgctctggtctatataaccacatttctgagcctttgagactgttcctgagccttcagcactaacctatgagggtgagctggtcccctctatatatacatcatacttaactttactaagtaatctcacagcatttgccaagtctcccaatatccaatt

SEQ ID NO: 10 - DNA, 497 bp, Artificial Sequence
  Affymetrix Probe ID No. 205147_x_at
agcagaggctctatttgacttcactggaaacagcaaactggagctgaatttcaaagctggagatgtgatcttcctcctcagtcggatcaacaaagactggctggagggcactgtccggggagccacgggcatcttccctctctccttcgtgaagatcctcaaagacttccctgaggaggacgaccccaccaactggctgcgttgctactactacgaagacaccatcagcaccatcaaggacatcgcggtggaggaagatctcagcagcactcccctattgaaagacctgctggagctcacaaggcgggagttccagagagaggacatagctctgaattaccgggacgctgagggggatctggttcggctgctgtcggatgaggacgtagcgctcatggtgcggcaggctcgtggcctcccctcccagaagcgcctcttcccctggaagctgcacatcacgcagaaggacaactacagggtctacaacacgatgccat

SEQ ID NO: 11 - DNA, 440 bp, Artificial Sequence
  Affymetrix Probe ID No. 223655_at
tctatgggactgtcacgccaaaccctggggacagagtgactgtggacacaaggaagatgctggcgtgaggtgctctggacagtcgctgaaatcactgaatgcctcctcaggtcgtttagcacttattttatccagtatctttgggctccttctcccggttctgtttattctatttctcacgtggtgccgagttcagaaacaaaaacatctgcccctcagagtttcaaccagaaggaggggttctctcgaggagaatttattccatgagatggagacctgcctcaagagagaggacccacatgggacaagaacctcagatgacacccccaaccatggttgtgaagatgctagcgacacatcgctgttgggagttcttcctgcctctgaagccacaaaatgactttagacttccagggctcaccagatcaacctctaaatat

SEQ ID NO: 12 - DNA, 482 bp, Artificial Sequence
  Affymetrix Probe ID No. 203104_at
tgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcattggctggccacaccaagcaggaagcacaaactcccccaagctgactcatcctaactaacagtcacgccgtg

SEQ ID NO: 13 - DNA, 515 bp, Artificial Sequence
  Affymetrix Probe ID No. 203561_at
tgctgggatgaccagcatcagccccaatgtccagcctctttaacatcttctttcctatgccctctctgtggatccctactgctggtttctgccttctccatgctgagaacaaaatcacctattcactgcttatgcagtcggaagctccagaagaacaaagagcccaattaccagaaccacattaagtctccattgttttgccttgggatttgagaagagaattagagaggtgaggatctggtatttcctggactaaattccccttggggaagacgaagggatgctgcagttccaaaagagaaggactcttccagagtcatctacctgagtcccaaagctccctgtcctgaaagccacagacaatatggtcccaaatgactgactgcaccttctgtgcctcagccgttcttgacatcaagaatcttctgttccacatccacacagccaatacaattagtcaaaccactgttattaacagatgtagcaacatgaaagacgctatgttacaggttaca

SEQ ID NO: 14 - DNA, 477 bp, Artificial Sequence
  Affymetrix Probe ID No. 205968_at
attgtggtgagcgatcctgactccacagatgcttcaagcattgaagacaatgaggacatttgtaacaccacctccttggagaattgcacagcaaaatgagcgggggtgtttgtgcctgtttctcttatcctttcccaacattaggttaacacagctttataaacctcagtgggttcgttaaaatcatttaattctcagggtgtacctttccagccatagttggacattcattgctgaattctgaaatgatagaattgtctttatttttctctgtgaggtcaattaaatgccttgttctgaaatttattttttacaagagagagttgtgatatagtttggaatataagataaatggtattgggtggggtttgtggctacagcttatgcatcattctgtgtttgtcatttactcacattgagctaactttaaattactgacaagtagaatcaaaggtgcagctgactgagacgacatgc

SEQ ID NO: 15 - DNA, 512 bp, Artificial Sequence
  Affymetrix Probe ID No. 204687_at
aatctctatttatctggttgtttctgacaggatgctgcctgcttggctctacaagctggaaagcagcttcttagctgcctaattaatgaaagatgaaaataggaagtgccctggagggggccagcaggtcacggggcagaatctctcaggttgctgtgggatctcagtgtgcccctacctgttctcccctccaggccacctgtctctgtaaaggatgtctgctctgttcaaaaggcagctgggatcccagcccacaagtgatcagcagagttgcatttccaaagaaaaaggctatgagatgagctgagttatagagagaaagggagaggcatgtacggtgtggggaagtggaagggaagctggcgggggagaaggaggctaacctgcactgagtacttcattaggacaagtgagaatcagctattgataatggccagagatatccacagcttggaggagcccagagaccgtttgctttatacccacacagcaactggtccactgctttactg

SEQ ID NO: 16 - DNA, 228 bp, Artificial Sequence
  Affymetrix Probe ID No. 229369_at
ggggtggcgcaaggagggaggaaagggcttgagttaaaagcgggtgcctgcaaccctcaaactccgacatcattcagtgtgtttaggggcaggaggtgttgttcagccgtggaatttgctggtggcagcagtgtaacctgtgtatttgagggtacaggcaancggtacagggtggagtggctggtccacaagctgtggcagggaagctgtttgcaggactgccctgcc

SEQ ID NO: 17 - protein, 310 aa, Homo sapiens
  misc_feature: Hs-NP-056208
MVYKTLFALCILTAGWRVQSLPTSAPLSVSLPTNIVPPTTIWTSSPQNTDADTASPSNGTHNNSVLPVTASAPTSLLPKNISIESREEEITSPGSNWEGTNTDPSPSGFSSTSGGVHLTTTLEEHSSGTPEAGVAATLSQSAAEPPTLISPQAPASSPSSLSTSPPEVFSASVTTNHSSTVTSTQPTGAPTAPESPTEESSSDHTPTSHATAEPVPQEKTPPTTVSGKVMCELIDMETTTTFPRVIMQEVEHALSSGSIAAITVTVIAVVLLVFGVAAYLKIRHSSYGRLLDDHDYGSWGNYNNPLYDDS

SEQ ID NO: 18 - protein, 296 aa, Mus musculus
  misc_feature: Mm_NP_663537
MVCKVLIALCIFTAGLRVQGSPTVPLPVSLMTKSSAPVATWTTSAPHTARATTPVASATHNASVLRTTAASLTSQLPTDHREEAVTSPPLKRDVNSTDSSPAGFPSTSSDGHLAPTPEEHSLGSPEATVPATGSQSPMLLSSQAPTSATTSPATSLSESLSASVTSSHNSTVANIQPTEAPMAPASPTEEHSSSHTPTSHVTAEPVPKEKSPQDTEPGKVICESETTTPFLIMQEVENALSSGSIAAITVTVIAVVLLVFGGAAYLKIRHSSYGRLLDDHDYGSWGNYNNPLYDDS

SEQ ID NO: 19 - protein, 298 aa, Rattus rattus
  misc_feature: Rn_NP_775137
MVCKALITLCIFAAGLMVQGSPTPTLLPVSLTTKSTAPMATWTTSAQHTAMATTPVASATHNASVLRTTAASLTSQLPTHPREEAVTSPPLKREVNSTDSSPTGFSSNSSGIHLAPTPEEHSLGSPETSVPATGSQSPTLLFSQGPTSASTSPATSPSEPLSASVTSNHSSTVNNIQPTGAPMAPASPTEEHSSSHTPTSHVTEPVPKEKSPQDTEPGKVICESETTTPFLIMQEVENALSSGSIAAITVTVIAVVLLVFGAAAYLKIRHSSYGRLLDDHDYGSGSWGNYNNPLYDDS